SEQUENCE LISTING (49 sequences)

SEQ ID NO: 1 - DNA, 984 bp, Pantoea ananatis
atgaataacaatttcacctttacgattaagagcagtgtttttgatgagaattacaatccgtcagaaaacacccgcatcacgaccaacttcgctaacctggcccgtggcaagaaccgccaggaaaacctgcgtaacgccctggtgatgatggataatcgttttaattccctggcgcactgggataacccgaaaggggaccgttattctgtcgagcttgagatcatttccgttgagatgaatatcaatgtggaaaataatcgcgatgctttcccggttatcgaaatattgaaaaccagtatcgtcgataaaaaaaccgacgaacgtattgaaggcattgtggggaacaacttctcttcttacgtgcgtgactatgacttcagcgtcttgctgtcagagcacaacaaaaataatccggtgtttactatcccggaaaactttggcgagctgcatggcaatatcttcaaatgcttcctgcaatcagaggcttacaaggcgaattttagcaagtcaccggtgatctgcctgagtgtttcaagcaaaaacacctatgtccgcaccggcaaccgtcacccggtactgggcattgagtaccagcaggatgaatattcgctgaccgataaatacttcagcaagatgggattacatgcgcgctattttatgcctgaaaacagcgttgcgcccctggcgttctattttacgggtgatctgctgcgtgattacactaatctcgaactcatcagcaccatcagtacgatggaaacgttccagaagatttatcgccctgagatctataatgcaaactcgccggctggtattgaatatcaaccgagcctgagccatcgggattattctttaacgcaaattgtttacgaccgagaagagcgtagccatctggctattgaacaagggaagtttaccgagaagaacttcatcaaaccctacaaaaatgttcttgagcaatggtctgctaattacgttctttga

SEQ ID NO: 2 - protein, 326 aa, Pantoea ananatis
MNNNFTFTIKSSVFDENYNPSENTRITTNFANLARGKNRQENLRNALVMMDNRFNSLAHWDNPKGDRYSVELEIISVEMNINVENNRDAFPVIEILKTSIVDKKTDERIEGIVGNNFSSYVRDYDFSVLLSEHNKNNPVFTIPENFGELHGNIFKCFLQSEAYKANFSKSPVICLSVSSKNTYVRTGNRHPVLGIEYQQDEYSLTDKYFSKMGLHARYFMPENSVAPLAFYFTGDLLRDYTNLELISTISTMETFQKIYRPEIYNANSPAGIEYQPSLSHRDYSLTQIVYDREERSHLAIEQGKFTEKNFIKPYKNVLEQWSANYV

SEQ ID NO: 3 - DNA, 1032 bp, Pantoea ananatis
atgaaaactttgttaccgacttccacggcgggaagcctgccaaaaccgtcctggcttgcgcaacctgaaacactgtggtcgccctggaaactggaaaatgaagaattaatcgatggcaagaaagatgcactgcgtttgattctggacgatcaggatcgcgccggtatcgatatcgtcagcgatggtgagcaaacgcgccaacactttgtgaccacctttattgaacaccttagcggtgtggattttgaaaaacgcgagatcgtcaaaatccgtaatcgttatgatgccagcgtgccgagtgtggtgggtgaagtggcccgtcagaagccggtcttcgttgaagatgccaaatttttacgtcagctgacgaataaacagatcaaatgggcgctgccgggtcccatgaccatgatcgatacgctgtatgacaaccattacaagagccgcgaaaaactggcctgggaatttgccaaaattcttaaccaggaagcgaaggaacttgaggcggcgggtgtcgatattatccagtttgacgaaccggcatttaacgtgtttttcgatgaggtgaatgactggggcattgccgccctggaacgcgccgttgaagggctcaagtgcgagaccgctgtgcacatttgctacggttacggcatcaaagccaacaccgactggaaaaagacgctcggaaccgaatggcgtcagtatgaagaagtcttccctaagctgcagaagtcgaacatcgatatcatctcgctggaatgccaaaactctcgcgtgccgatggatcttatcgaactgattcgcggtaaaaaagtgatggtcggggccattgatgtggcaaccaacgcgatcgaaacccctgaagaagtggccgatacgctgcgtaaagcccttcagtttgtggatgccgataagctttatccctctaccaactgtggcatgacgccgttatcccgtcaggttgcccggggcaagctgagtgccctcagcgccggtgcagaaatcatccgtaaagaactgctgtcgaaataa

SEQ ID NO: 4 - protein, 343 aa, Pantoea ananatis
MKTLLPTSTAGSLPKPSWLAQPETLWSPWKLENEELIDGKKDALRLILDDQDRAGIDIVSDGEQTRQHFVTTFIEHLSGVDFEKREIVKIRNRYDASVPSVVGEVARQKPVFVEDAKFLRQLTNKQIKWALPGPMTMIDTLYDNHYKSREKLAWEFAKILNQEAKELEAAGVDIIQFDEPAFNVFFDEVNDWGIAALERAVEGLKCETAVHICYGYGIKANTDWKKTLGTEWRQYEEVFPKLQKSNIDIISLECQNSRVPMDLIELIRGKKVMVGAIDVATNAIETPEEVADTLRKALQFVDADKLYPSTNCGMTPLSRQVARGKLSALSAGAEIIRKELLSK

SEQ ID NO: 5 - DNA, 882 bp, Pantoea ananatis
gtgaccactctggaagacaccatcggcaatacgccgctcatcagattacagcgcctggcgcctgacaacggcagtgaagtctggcttaagctggaaggcaataaccctgccggttcagtgaaagatcgggccgcctggtcgatgatccaccaggccgagttgcgcggcgacattacgccgggcgaccagttgatcgaagccaccagtggcaataccggcattgcgctggcgatgattgccgcgatgaaggggtatcggctgcggctgctgatgccagacaacatgagtcaggagcggcaggatgcgatgcgcgcctacggcgcagaactggtgctgattagccgcgagcaggggatggaaggcgcacgcgatcgggctcaggagatggcggcacggggcgaaggaagggtactggaccagttcaataacccggataacccgctcggccattaccaaaccaccgggcctgagctgtggcagcagtcgaaacagcgaatgacccactttgtttccagcatgggtaccacgggaaccattactggcgtaggccggtttttaaaggatcaccagaccggcgttaaagtgattggcctgcagcctaccgaaggcagcagcattcccggcatccgccgctggccccaggcttacatgccgggcatttatcgccctgagttggtggatgacatcattgatatgggacagcaggaagccgaagaaaccatgcgcgcgttagcgcgacgtgaaggcatcttctgcggcgtcagttccggcggttcggttgctggcgcactgcgcatcgcccgggcgaatccggggagtgtggtggtggctatcgtctgcgaccggggcgatcgctatctctcaaccggaatttatcagtag

SEQ ID NO: 6 - DNA, 66 bp, Artificial Sequence
  Primer P1
ggctgcagcaggccagactgtaccagggcgcgacaccgtgaagcctgcttttttatactaagttgg

SEQ ID NO: 7 - DNA, 67 bp, Artificial Sequence
  Primer P2
agcggcgtattgccgatggtgtcttccagagtggtcatttatcatccaggaaaaattggttaataac

SEQ ID NO: 8 - DNA, 5743 bp, Artificial Sequence
  pMW118-attL-kan-attR-Pnlp8sd22 plasmid
gacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttaggtaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcccattacatggggatcttgaagcctgcttttttatactaagttggcattataaaaaagcattgcttatcaatttgttgcaacgaacaggtcactatcagtccaaataaaatcattatttgatttcgtcgagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcagcaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccctgcagtctgttacaggtcactaataccatctaagtagttgattcatagtgactgcatatgttgtgttttacagtattatgtagtctgttttttatgcaaaatctaatttaatatattgatatttatatcattttacgtttaccgttcagcttttttatactaacttgctgcgctaatgacgcagctggacgaaggcgggattctcgtcttacccgtaggggaggagcaccagtatttgaaacgggtgcgtcgtcggggaggcgaatttattatcgataccgtggaggccgtgcgctttgtccctttagtgaagggtgagctggcttaaaacgtgaggaaatacctggatttttcctggttattttgccgcaggtcagcgtataatgaagatcttttccagtgttgacaagggtccttgcacggttataatgtcactggttattaaccaatttttcctggatgataatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 9 - DNA, 20 bp, Artificial Sequence
  Primer P3
tggtctttgacggtgaacag

SEQ ID NO: 10 - DNA, 20 bp, Artificial Sequence
  Primer P4
ccttccagcttaagccagac

SEQ ID NO: 11 - DNA, 1284 bp, Pantoea ananatis
atgacttcaccctcaagtaagaaaacgcacgttggcgatcgtcaactgcatcctgaaactcagatgctgaattatggctacgatcctcgcctgtcggaaggtgccgtgaagccgcccgtgtttcttacctctacctttgtttttgacagcgcagaagaaggccgcgattttttcgactatgtttcaggacgtcgtcagcctccggaaggcaaaagcggcggactggtttattcccgctttaatcatcccaacagcgagatcgtagaagatcgcctggccatttacgaacaggcggaaagctgtgccctgtttgcttccggtatggcggctatctcgacaacgctgttaacctttgcacgaccgggagaagtcattctgcattcgcagccgctgtatggcggcacggaaacgttgctgagtaagacactgcacaatctggacattcaggcggtcggcttcagcgacggacacgatgaagcaaaagtgcgtgatgcggctcatcgtgcgatggcgcaggggcgcgtcgcgatgattctggtggaaaccccggcgaatcccaccaacagcctggtgaatattgcgctgatggggcgcatcgccgacgagatcgatcggcatcagggttcgcgtccggtcatcgcctgtgataacaccttgctgggcccgatttttcaacagccgctgacgctgggggcggacttatcgctgtattctctgacgaaatatgtcggcgggcactctgacctgatcgctggcgcggtgatgggtaagaaaacgctgatgacgcaaattcgcgccctgcgaagcgctatcggtacgcaactggatccgcactcaagctggatgattagccgttcgctggaaacgctgtcgctgcgcatggaaaaagcctttcgcaatgccgaagccgttgccgcctttctgcagccgcatcctaaggtagcagcgttgcactggttgccctatctggatgcagaaagccccgcaggcaaaacgtatcgcgagcagtgcagcggcgccggctcgaccttctcttttgatatcgtgggcggacaacctgccgcgtttcgctttctcaatgcgcttgcgttgtttaagcaggccgttagcctgggcggcaccgagtcacttgccagccatcccggcacaaccacgcattctggcgtgccggatgcagtccgtgagcgcatcggtattaccgacgcgacgatacgcttatcgatcggcatcgaacatgccggagatctggtagaagacatccgactggcccttgaacaagcctga

SEQ ID NO: 12 - DNA, 60 bp, Artificial Sequence
  Primer P5
agccgcccgtgtttcttacctctacctttgtttttgtgaagcctgcttttttatactaag

SEQ ID NO: 13 - DNA, 59 bp, Artificial Sequence
  Primer P6
agataccgggatgatgctgggcttcaccgtgacctggctactgataaattccggttgag

SEQ ID NO: 14 - DNA, 21 bp, Artificial Sequence
  Primer P7
tgcagttaacgaccttattgg

SEQ ID NO: 15 - DNA, 20 bp, Artificial Sequence
  Primer P8
gtactcctgatgatgcatgg

SEQ ID NO: 16 - DNA, 318 bp, Pantoea ananatis
atggctgaatggaacggcgaatatatcagcccgtacgctgagcacggcaaaaagagcgaacaggttaaaaaaatcacggtatcaattccgttaagcgtgctgaaaattctgacagacgaacggacacgtcgtcaggttaacaacctgcgccacgcgacaaacagtgaactcctgtgcgaagccttcttgcatgcttttaccggacaaccgttacctaatgatgtcgacctgcgtaaagaacgtaacgacgaaataccggaagccgcgaaaaaaattatgcgtgacatgggcatcgacccggatacctgggaatactga

SEQ ID NO: 17 - DNA, 60 bp, Artificial Sequence
  Primer P9
taacttttaacttttattgattaaggtatcccatggtgaagcctgcttttttatactaag

SEQ ID NO: 18 - DNA, 60 bp, Artificial Sequence
  Primer P10
cagctcaggctgggtttttttatgtctgaaaatcagcgctcaagttagtataaaaaagct

SEQ ID NO: 19 - DNA, 20 bp, Artificial Sequence
  Primer P11
gtgcgttgaacgaagaaacc

SEQ ID NO: 20 - DNA, 20 bp, Artificial Sequence
  Primer P12
cgcttcaacaagcgtttcag

SEQ ID NO: 21 - DNA, 930 bp, Pantoea ananatis
atgccaatcagggtccccgacgaattaccggcagtgaattttttacgtaacgaaaacgtctttgtgatgacgtcgtcacgtgccagtgttcaggagatccgtccgcttaaagtgctggttcttaatctcatgcccaagaagattgagacagaaaaccagtttctgcgcttactctctaactcaccgctgcagatcgatatccagctactgcgcatcgacagccgggagtcgcggaatacacccagcgatcatctcaacaacttctactgcaattttaacgatattcagcacgataactacgatggcttaatcgtgacaggtgcgccgctgggcctggttgaatttaacgatgtggcttactggccgcagataaaacaggtgctggagtgggcgaaagatcatgttacctccacgctgtttgtctgttgggccgtccaggcggcattaaacattttatatggcattcctaaacagacccggcagactaaactggcgggcgtctttgagcatcagatactgcatccccattccttactgacgcggggattcgacgataatttcctcgctccccattcacgttacgctgatttcccgacgcagctcatcacggattataccgatctggaattatttgctgaatcggaacaaaccggcgcgtatctgatggcgagcaaagacaaaaggctggcgtttgtgaccgggcatccggagtacgatgcactgacgctggcgggagagtttcaacgcgactatgaagccgggctgaatcctgaagtaccctataactatttcccacaggataatccgcagcttacaccgcgcgccacctggcgtagccatggaaacctgctgttttcaaactggctgaattattacgtctaccagattacgccgtttgatctgcgccatatgaatcccacgctggaataa

SEQ ID NO: 22 - DNA, 60 bp, Artificial Sequence
  Primer P13
gacagaacaggctttcaatttcttttccgcaggggctgaagcctgcttttttatactaag

SEQ ID NO: 23 - DNA, 60 bp, Artificial Sequence
  Primer P14
cactgccggtaattcgtcggggaccctgattggcattatatctccttcttaaagttaaac

SEQ ID NO: 24 - DNA, 5533 bp, Artificial Sequence
  pMW118-attL-kan-attR-Ptac71phi10 plasmid
gacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttaggtaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcccattacatggggatcttgaagcctgcttttttatactaagttggcattataaaaaagcattgcttatcaatttgttgcaacgaacaggtcactatcagtccaaataaaatcattatttgatttcgtcgagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcagcaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccctgcagtctgttacaggtcactaataccatctaagtagttgattcatagtgactgcatatgttgtgttttacagtattatgtagtctgttttttatgcaaaatctaatttaatatattgatatttatatcattttacgtttaccgttcagcttttttatactaacttgagatctccctgttgacaattaatcatcggctcgtataatgtgtggaatcgtgagcggataacaatttcacactttaagaaggagatatatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 25 - DNA, 20 bp, Artificial Sequence
  Primer P15
cgccagtacggttttctgtc

SEQ ID NO: 26 - DNA, 22 bp, Artificial Sequence
  Primer P16
ttttctgtctcaatcttcttgg

SEQ ID NO: 27 - protein, 309 aa, Pantoea ananatis
MPIRVPDELPAVNFLRNENVFVMTSSRASVQEIRPLKVLVLNLMPKKIETENQFLRLLSNSPLQIDIQLLRIDSRESRNTPSDHLNNFYCNFNDIQHDNYDGLIVTGAPLGLVEFNDVAYWPQIKQVLEWAKDHVTSTLFVCWAVQAALNILYGIPKQTRQTKLAGVFEHQILHPHSLLTRGFDDNFLAPHSRYADFPTQLITDYTDLELFAESEQTGAYLMASKDKRLAFVTGHPEYDALTLAGEFQRDYEAGLNPEVPYNYFPQDNPQLTPRATWRSHGNLLFSNWLNYYVYQITPFDLRHMNPTLE

SEQ ID NO: 28 - DNA, 930 bp, Artificial Sequence
  mutant gene metA
atgccaatcagggtccccgacgaattaccggcagtgaattttttacgtaacgaaaacgtctttgtgatgacgtcgtcacgtgccagtgttcaggagatctgtccgcttaaagtgctggttcttaatctcatgcccaagaagattgagacagaaaaccagtttctgcgcttactctctaactcaccgctgcagatcgatatccagctactgcgcatcgacagccgggagtcgcggaatacacccagcgatcatctcaacaacttctactgcaattttaacgatattcagcacgataactacgatggcttaatcgtgacaggtgcgccgctgggcctggttgaatttaacgatgtggcttactggccgcagataaaacaggtgctggagtgggcgaaagatcatgttacctccacgctgtttgtctgttgggccgtccaggcggcattaaacattttatatggcattcctaaacagacccggcagactaaactggcgggcgtctttgagcatcagatactgcatccccattccttactgacgcggggattcgacgataatttcctcgctccccattcacgttacgctgatttcccgacgcagctcatcacggattataccgatctggaattatttgctgaatcggaacaaaccggcgcgtatctgatggcgagcaaagacaaaaggctggcgtttgtgaccgggcatccggagtacgatgcactgacgctggcgggagagtttcaacgcgactatgaagccgggctgaatcctgaagtaccctataactatttcccacaggataatccgcagcttacaccgcgcgccacctggcgtagccatggaaacctgctgttttcaaactggctgaattattacgtctaccagattacgccgtttgatctgcgccatatgaatcccacgctggaataa

SEQ ID NO: 29 - protein, 309 aa, Artificial Sequence
  mutant protein MetA (R34C)
MPIRVPDELPAVNFLRNENVFVMTSSRASVQEICPLKVLVLNLMPKKIETENQFLRLLSNSPLQIDIQLLRIDSRESRNTPSDHLNNFYCNFNDIQHDNYDGLIVTGAPLGLVEFNDVAYWPQIKQVLEWAKDHVTSTLFVCWAVQAALNILYGIPKQTRQTKLAGVFEHQILHPHSLLTRGFDDNFLAPHSRYADFPTQLITDYTDLELFAESEQTGAYLMASKDKRLAFVTGHPEYDALTLAGEFQRDYEAGLNPEVPYNYFPQDNPQLTPRATWRSHGNLLFSNWLNYYVYQITPFDLRHMNPTLE

SEQ ID NO: 30 - DNA, 64 bp, Artificial Sequence
  Primer P17
atgttaatgtgaaattaagtcgctttcactcatgtttgaagcctgcttttttatactaagttgg

SEQ ID NO: 31 - DNA, 59 bp, Artificial Sequence
  Primer P18
actgctcttaatcgtaaaggtgaaattgttattcattatatctccttcttaaagtgtga

SEQ ID NO: 32 - DNA, 5620 bp, Artificial Sequence
  pMW118-attL-cat-attR-Ptac71phi10 plasmid
gacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttaggtaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcccattacatggggatcttgaagcctgcttttttatactaagttggcattataaaaaagcattgcttatcaatttgttgcaacgaacaggtcactatcagtccaaataaaatcattatttgatttcgtcgagttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagctgcagtctgttacaggtcactaataccatctaagtagttgattcatagtgactgcatatgttgtgttttacagtattatgtagtctgttttttatgcaaaatctaatttaatatattgatatttatatcattttacgtttaccgttcagcttttttatactaacttgagatctccctgttgacaattaatcatcggctcgtataatgtgtggaatcgtgagcggataacaatttcacactttaagaaggagatatatctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 33 - DNA, 21 bp, Artificial Sequence
  Primer P19
atgggttcgtacagcaacgga

SEQ ID NO: 34 - DNA, 21 bp, Artificial Sequence
  Primer P20
caggttagcgaagttggtcgt

SEQ ID NO: 35 - DNA, 906 bp, Pantoea ananatis
atgaatcgaacactggcggacctggatcgcgtggtggccttaggtggcggacacggattaggacgcgtgatgtcggccctgtcccctctgggttcacgcctcacaggcattgtgaccacgacagacaacggtggctccacgggccgcattcgtcgcgcagaaggtggcatcgcctggggcgacctgcgtaactgtattaaccagctgatcacggagccgagcgtggcctccgccatgttcgaatatcgctttaccggcaacggtgaactggccggacacaatctgggtaacctgatactgaaagcgcttgatcaccttagcgtgcggccgctggaagtggtcaacatcatcagaaacctgttgaaagttgacgcctttttaatccccatgtcagagcatccggttgatttagttgcaaccgatagcgagggcaacgcggtgtatggggaaaccgctatcgacgccatgcaacttccaccgcaggaactgatgctgcatcctgccgtgcagcccacccgcgaagcgcttgatgccgttgcagaagccgatctcattctgattggaccgggcagcttttacaccagcctgatgcccattctgctcatgaaagagatggcgcaggcattacgccggacgcccgctaacatggtgtttatcggcaacctgggccgcgagttgagtcctgcggcggcaacgcttaccgtggccgacaaactggcgatgatggagcactacattggcaagcgggtgatcgacgcgatcgtagttggcccgtccacggacgtcagccgggtggaaaaccgcctgattattcgtgaaccgctggaggccgccgatatcaagtaccgccacgaccgccaactgctgcgcgccgcgctggagcacgccgttcagggattcaactga

SEQ ID NO: 36 - DNA, 64 bp, Artificial Sequence
  Primer P21
tttaccggcaacggtgaactggccggacacaatctgtgaagcctgcttttttatactaagttgg

SEQ ID NO: 37 - DNA, 60 bp, Artificial Sequence
  Primer P22
aggaattgcgcacattgtttattgcggtgtcctgatttatttcgacagcagttctttacg

SEQ ID NO: 38 - DNA, 20 bp, Artificial Sequence
  Primer P23
ggattaggacgcgtgatgtc

SEQ ID NO: 39 - DNA, 20 bp, Artificial Sequence
  Primer P24
cctggaactgggcaataacg

SEQ ID NO: 40 - DNA, 33 bp, Artificial Sequence
  Primer P25
agctgagcatgcaaaacgtgaggaaatacctgg

SEQ ID NO: 41 - DNA, 34 bp, Artificial Sequence
  Primer P26
agctgagtcgacggagcaccagtatttgaaacgg

SEQ ID NO: 42 - DNA, 31 bp, Artificial Sequence
  Primer P27
agctgagtcgacatggatacccagcaaaccc

SEQ ID NO: 43 - DNA, 32 bp, Artificial Sequence
  Primer P28
agctgatctagatttcacgctttgcgcattcg

SEQ ID NO: 44 - DNA, 900 bp, Pantoea ananatis
atggatacccagcaaacccgtcagggcatcggctttgcgcttggcgcttactttatttggggcattgcgcccgcctacttcaaattagtgaaggccgtaccggcgactgaaatcatgacccacagggtgatctggtcagcactgttcatgttgttactgattaccctgagtcgcagttggggacaggtcagaagcgtgctgcgccagccaaaaaaagtgctgctactggcccttacggcggtaacggtagggggaaactggttgctgtttatctgggcggtgaacaatcagcatatgcttgaagccagcctgggctattttatcaacccgttgattaacgttgtgtttggcatgattttcctgcgtgaacgtttccgtcgcctgcagtggctggcggtcgccctggcggctggcggcgtgcttctgcaactgtggaaatttggctcagtgccggtgattgccttagggctggcgttcagcttcgcgttttatggcctgatgaggaagaaaattcaggtcgatgcccagagcggcatgctgatagagacgctgtggctgtttccgctggccgcgctctatctgtttggctttgctgacagcagcaccagccaccttagcgcgaaccccatgagcctgaacctgttactgatcgccgcaggcattgttaccacgattccgctgatgttcttcgccgcggcttgtacccgactgcgcctgtcaacggtgggcttctttcagtatctgggccctacgctgatgttcttgctggcggtgctgttttatggtgaaaccgttacgccagataaaatggtaacctttggttttatctggctggctctgctggtctttattctggatgccgtggcgttttcagcgcggagccgaatgcgcaaagcgtga

SEQ ID NO: 45 - protein, 299 aa, Pantoea ananatis
MDTQQTRQGIGFALGAYFIWGIAPAYFKLVKAVPATEIMTHRVIWSALFMLLLITLSRSWGQVRSVLRQPKKVLLLALTAVTVGGNWLLFIWAVNNQHMLEASLGYFINPLINVVFGMIFLRERFRRLQWLAVALAAGGVLLQLWKFGSVPVIALGLAFSFAFYGLMRKKIQVDAQSGMLIETLWLFPLAALYLFGFADSSTSHLSANPMSLNLLLIAAGIVTTIPLMFFAAACTRLRLSTVGFFQYLGPTLMFLLAVLFYGETVTPDKMVTFGFIWLALLVFILDAVAFSARSRMRKA

SEQ ID NO: 46 - DNA, 900 bp, Artificial Sequence
  mutant gene rarD
atggatacccagcaaacccgtcagggcatcggctttgcgcttggcgcttactttatttggggcattgcgcccgcctacttcaaattagtgaaggccgtaccggcgactgaaatcatgacccacagggtgatctggtcagcactgttcatgttgttactgattaccctgagtcgcagttggggacaggtcagaagcgtgctgcgccagccaaaaaaagtgctgctactggcccttacggcggtaacggtagggggagactggttgctgtttatctgggcggtgaacaatcagcatatgcttgaagccagcctgggctattttatcaacccgttgattaacgttgtgtttggcatgattttcctgcgtgaacgtttccgtcgcctgcagtggctggcggtcgccctggcggctggcggcgtgcttctgcaactgtggaaatttggctcagtgccggtgattgccttagggctggcgttcagcttcgcgttttatggcctgatgaggaagaaaattcaggtcgatgcccagagcggcatgctgatagagacgctgtggctgtttccgctggccgcgctctatctgtttggctttgctgacagcagcaccagccaccttagcgcgaaccccatgagcctgaacctgttactgatcgccgcaggcattgttaccacgattccgctgatgttcttcgccgcggcttgtacccgactgcgcctgtcaacggtgggcttctttcagtatctgggccctacgctgatgttcttgctggcggtgctgttttatggtgaaaccgttacgccagataaaatggtaacctttggttttatctggctggctctgctggtctttattctggatgccgtggcgttttcagcgcggagccgaatgcgcaaagcgtga

SEQ ID NO: 47 - protein, 299 aa, Artificial Sequence
  mutant protein RarD (N86D)
MDTQQTRQGIGFALGAYFIWGIAPAYFKLVKAVPATEIMTHRVIWSALFMLLLITLSRSWGQVRSVLRQPKKVLLLALTAVTVGGDWLLFIWAVNNQHMLEASLGYFINPLINVVFGMIFLRERFRRLQWLAVALAAGGVLLQLWKFGSVPVIALGLAFSFAFYGLMRKKIQVDAQSGMLIETLWLFPLAALYLFGFADSSTSHLSANPMSLNLLLIAAGIVTTIPLMFFAAACTRLRLSTVGFFQYLGPTLMFLLAVLFYGETVTPDKMVTFGFIWLALLVFILDAVAFSARSRMRKA

SEQ ID NO: 48 - protein, 293 aa, Pantoea ananatis
MTTLEDTIGNTPLIRLQRLAPDNGSEVWLKLEGNNPAGSVKDRAAWSMIHQAELRGDITPGDQLIEATSGNTGIALAMIAAMKGYRLRLLMPDNMSQERQDAMRAYGAELVLISREQGMEGARDRAQEMAARGEGRVLDQFNNPDNPLGHYQTTGPELWQQSKQRMTHFVSSMGTTGTITGVGRFLKDHQTGVKVIGLQPTEGSSIPGIRRWPQAYMPGIYRPELVDDIIDMGQQEAEETMRALARREGIFCGVSSGGSVAGALRIARANPGSVVVAIVCDRGDRYLSTGIYQ

SEQ ID NO: 49 - protein, 105 aa, Pantoea ananatis
MAEWNGEYISPYAEHGKKSEQVKKITVSIPLSVLKILTDERTRRQVNNLRHATNSELLCEAFLHAFTGQPLPNDVDLRKERNDEIPEAAKKIMRDMGIDPDTWEY

(19) United States
(12) Patent Application Publication
(10) Pub. No.: US 2021/0207182 A1
(43) Pub. Date: Jul. 8, 2021

(54) METHOD FOR PRODUCING L-METHIONINE USING A BACTERIUM

(71) Applicant: AJINOMOTO CO., INC., Tokyo (JP)

(72) Inventors: Mikhail Kharisovich Ziyatdinov, Moscow (RU); Andrey Olegovich Lobanov, Moscow (RU)

(73) Assignee: AJINOMOTO CO., INC., Tokyo (JP)

(21) Appl. No.: 17/211,479

(22) Filed: Mar. 24, 2021

Related U.S. Application Data
(63) Continuation of application No. PCT/JP2019/038308, filed on Sep. 27, 2019.

(30) Foreign Application Priority Data
Sep. 28, 2018 (RU) 2018134156

(51) Int. Cl. C12P 13/12

(52) U.S. Cl. CPC C12P 13/12; C12Y 203/01046; C12Y 205/01047

(57) ABSTRACT

A method is described for producing L-methionine by fermentation using a bacterium which has been modified to overexpress a 1st gene and a 2nd gene, such as genes having the nucleotide sequences shown in SEQ ID NOs: 1 and 3.

This application is a Continuation of, and claims priority under 35 U.S.C. § 120 to, International Application No. PCT/JP2019/038308, filed Sep. 27, 2019, and claims priority therethrough under 35 U.S.C. § 119 to Russian Patent Application No. 2018134156, filed Sep. 28, 2018, the entireties of which are incorporated by reference herein. Also, the Sequence Listing filed electronically herewith is hereby incorporated by reference (File name: 2021-03-24T_US-628_Seq_List; File size: 58 KB; Date recorded: Mar. 24, 2021).

### BACKGROUND

General Field

The present invention relates generally to the microbiological industry, and specifically to a method for producing L-methionine by fermentation of a bacterium which has been modified to overexpress a 1st gene and a 2nd gene as described herein, such as genes having the nucleotide sequences shown in SEQ ID NOs: 1 and 3, so that production of the L-methionine is enhanced as compared with a non-modified bacterium.

### DESCRIPTION OF THE RELATED ART

Conventionally, L-amino acids are industrially produced by fermentation methods utilizing strains of microorganisms obtained from natural sources, or mutants thereof. Typically, the microorganisms are modified to enhance production yields of L-amino acids.

Many techniques to enhance L-amino acids production yields have been reported, including transformation of microorganisms with recombinant DNA (see, for example, U.S. Pat. No. 4,278,765 A) and alteration of expression regulatory regions such as promoters, leader sequences, and/or attenuators, or others known to the person skilled in the art (see, for example, US20060216796 A1 and WO9615246 A1). Other techniques for enhancing production yields include increasing the activities of enzymes involved in amino acid biosynthesis and/or desensitizing the target enzymes to the feedback inhibition by the resulting L-amino acid (see, for example, WO9516042 A1, EP0685555 A1 or U.S. Pat. Nos. 4,346,170 A, 5,661,012 A, and 6,040,160 A).

Another method for enhancing L-amino acids production yields is to attenuate expression of a gene or several genes which are involved in degradation of the target L-amino acid, genes which divert the precursors of the target L-amino acid from the L-amino acid biosynthetic pathway, genes involved in the redistribution of the carbon, nitrogen, sulfur, and phosphate fluxes, and genes encoding toxins, etc.

As for L-methionine (also known as (2S)-2-amino-4-(methylsulfanyl) butanoic acid), a method for producing L-methionine by culturing in a medium a recombinant Escherichia bacterium that is deficient, at least, in a repressor of the L-methionine biosynthesis system encoded by the metJ gene, is known (U.S. Pat. No. 7,611,873 B1). The bacterium used in the method was modified further to increase activity of intracellular homoserine transsuccinylase (MetA). Moreover, the homoserine transsuccinylase of the bacterium that was used in the method for producing L-methionine has been modified to render it insensitive to the feedback inhibition by L-methionine. Specifically, the amino acid sequence of the MetA in the Escherichia bacterium was modified to contain, at least, a substitution selected from the replacement of the arginine (Arg) residue at position 27 with cysteine (Cys) residue (R27C mutation), the isoleucine (Ile) residue at position 296 with serine (Ser) residue (I296S mutation), and the proline (Pro) residue at position 298 with leucine (Leu) residue (P298L mutation).

In another example, a recombinant microorganism, which may be a bacterium belonging to the family Enterobacteriaceae or Corynebacteriaceae, was modified in order to improve the fermentative production of L-methionine, such that the activity of the cobalamin-independent methionine synthase (MetE) is suppressed and the metH gene is overexpressed in the microorganism (EP2861726 B1; as for the use of enhanced 5-methyltetrahydrofolate homocysteine methyltransferase (MetH), see also, for example, WO0210209 A1).

Other methods for producing L-methionine by fermentation of a bacterium are known, including, for example, the method in which a bacterial strain having the ability to produce L-threonine and transformed with vector(s) expressing threonine dehydratase (tdcB, ilvA) and, at least, O-succinylhomoserine lyase (metB), cystathionine beta-lyase (metC), 5,10-methylenetetrahydrofolate reductase (metF) and serine hydroxymethyltransferase (glyA) was used (U.S. Pat. No. 7,790,424 B2); the method in which a microorganism of the Enterobacteriaceae family modified to enhance the transhydrogenase activity of PntAB was used (EP2633037 B1); and so forth.

A method for producing an L-amino acid has also been disclosed using a microorganism in which activity of a RarD protein native to an Escherichia coli (E. coli) species or an 80% or higher homologous variant thereof is enhanced, wherein the microorganism belongs to the genus Escherichia, Corynebacterium, Bacillus, Serratia, Pseudomonas or Streptomyces, and wherein the L-amino acid is, particularly, L-serine, L-glutamine, L-cysteine, L-phenylalanine, and L-threonine (US2012015409 A1). The RarD protein (synonym: YigH) native to E. coli is characterized as a putative member of the drug/metabolite transporter superfamily (EcoCyc database, ecocyc.org, accession ID: EG11466). In the Transporter Classification Database, RarD is classified as a member of the Chloramphenicol-Sensitivity Protein (RarD) Family within the Drug/Metabolite Transporter (DMT) superfamily (Saier M. H. Jr. et al., The Transporter Classification Database (TCDB): recent advances, Nucleic Acids Res., 2016, 44(D1):D372-9; doi: 10.1093/nar/gkv1103).

A gene having the nucleotide sequence shown in SEQ ID NO: 1 native to P. ananatis has the accession No. PAJ_RS05335 in the BioCyc database (biocyc.org). This gene encodes a hypothetical protein with unknown activity or function.

A gene having the nucleotide sequence shown in SEQ ID NO: 3 native to P. ananatis has the accession No. PAJ_RS05340 in the BioCyc database. This gene encodes a putative 5-methyltetrahydropteroyltriglutamate-homocysteine methyltransferase (Enzyme Commission (EC) No.: 2.1.1.14).

However, no method for producing L-methionine by fermentation of an L-methionine-producing bacterium that has been modified to overexpress a 1st gene and a 2nd gene as described herein, such as genes having the nucleotide sequences shown in SEQ ID NOs: 1 and 3, has been previously reported.

### SUMMARY

An improved method of producing L-methionine by fermentation of a bacterium is described herein. According to the presently disclosed subject matter, production of L-methionine by fermentation of a bacterium can be increased. Specifically, production of L-methionine by fermentation of a bacterium can be improved by overexpressing a 1st gene and a 2nd gene as described herein, such as genes having the nucleotide sequences shown in SEQ ID NOs: 1 and 3, in the bacterium, so that the production of L-methionine by the modified bacterium can be enhanced as compared with a non-modified bacterium.

The present invention thus provides the following:

It is an aspect of the invention to provide a method for producing L-methionine comprising:

(i) cultivating in a culture medium a bacterium which has an ability to produce L-methionine to produce and accumulate the L-methionine in the culture medium or the cells of the bacterium, or both, and

(ii) collecting the L-methionine from the culture medium or the cells, or both,

wherein said bacterium has been modified to overexpress a 1st gene and a 2nd gene,

wherein said 1st gene is selected from the group consisting of:

(1A) a DNA comprising the nucleotide sequence shown in SEQ ID NO: 1,

(1B) a DNA encoding a protein comprising the amino acid sequence shown in SEQ ID NO: 2,

(1C) a DNA encoding a protein comprising the amino acid sequence shown in SEQ ID NO: 2, but wherein said amino acid sequence includes substitution, deletion, insertion, and/or addition of 1 to 30 amino acid residues, and wherein said DNA comprises a property that if the DNA is overexpressed in the bacterium, the amount of L-methionine produced by the bacterium is increased as compared with that observed for a non-modified strain,

(1D) a DNA encoding a protein comprising an amino acid sequence having an identity of not less than 85% with respect to the entire amino acid sequence shown in SEQ ID NO: 2, and wherein said DNA comprises a property that if the DNA is overexpressed in the bacterium, the amount of L-methionine produced by the bacterium is increased as compared with that observed for a non-modified strain,

(1E) a DNA comprising a nucleotide sequence that is able to hybridize under stringent conditions with a nucleotide sequence complementary to the sequence shown in SEQ ID NO: 1, and wherein said DNA comprises a property that if the DNA is overexpressed in the bacterium, the amount of L-methionine produced by the bacterium is increased as compared with that observed for a non-modified strain,

(1F) a DNA comprising a nucleotide sequence having an identity of not less than 85% with respect to the entire nucleotide sequence shown in SEQ ID NO: 1, and wherein said DNA comprises a property that if the DNA is overexpressed in the bacterium, the amount of L-methionine produced by the bacterium is increased as compared with that observed for a non-modified strain, and

(1G) a DNA comprising a variant nucleotide sequence of SEQ ID NO: 1 due to the degeneracy of the genetic code; and

wherein said 2nd gene is selected from the group consisting of:

(2A) a DNA comprising the nucleotide sequence shown in SEQ ID NO: 3,

(2B) a DNA encoding a protein comprising the amino acid sequence shown in SEQ ID NO: 4,

(2C) a DNA encoding a protein comprising the amino acid sequence shown in SEQ ID NO: 4, but wherein said amino acid sequence includes substitution, deletion, insertion, and/or addition of 1 to 30 amino acid residues, and wherein said DNA comprises a property that if the DNA is overexpressed in the bacterium, the amount of L-methionine produced by the bacterium is increased as compared with that observed for a non-modified strain,

(2D) a DNA encoding a protein comprising an amino acid sequence having an identity of not less than 85% with respect to the entire amino acid sequence shown in SEQ ID NO: 4, and wherein said DNA comprises a property that if the DNA is overexpressed in the bacterium, the amount of L-methionine produced by the bacterium is increased as compared with that observed for a non-modified strain,

(2E) a DNA comprising a nucleotide sequence that is able to hybridize under stringent conditions with a nucleotide sequence complementary to the sequence shown in SEQ ID NO: 3, and wherein said DNA comprises a property that if the DNA is overexpressed in the bacterium, the amount of L-methionine produced by the bacterium is increased as compared with that observed for a non-modified strain,

(2F) a DNA comprising a nucleotide sequence having an identity of not less than 85% with respect to the entire nucleotide sequence shown in SEQ ID NO: 3, and wherein said DNA comprises a property that if the DNA is overexpressed in the bacterium, the amount of L-methionine produced by the bacterium is increased as compared with that observed for a non-modified strain, and

(2G) a DNA comprising a variant nucleotide sequence of SEQ ID NO: 3 due to the degeneracy of the genetic code.

It is another aspect of the invention to provide the method as described above, wherein each of said 1st and 2nd genes is overexpressed by increasing the copy number of the gene, by modifying an expression regulatory region of the gene, or a combination thereof, so that the expression of said 1st and 2nd genes is enhanced as compared with a non-modified bacterium.

It is another aspect of the invention to provide the method as described above, wherein said bacterium is a bacterium belonging to the family Enterobacteriaceae.

It is another aspect of the invention to provide the method as described above, wherein said bacterium is a bacterium belonging to the genus Escherichia or Pantoea.

It is another aspect of the invention to provide the method as described above, wherein said bacterium is Escherichia coli or Pantoea ananatis.

It is another aspect of the invention to provide the method as described above, wherein said bacterium has been further modified to overexpress a rarD gene.

It is another aspect of the invention to provide the method as described above, wherein said rarD gene is overexpressed by increasing the copy number of the gene, by modifying an expression regulatory region of the gene, or a combination thereof, so that the expression of said rarD gene is enhanced as compared with a non-modified bacterium.

It is another aspect of the invention to provide the method as described above, wherein said bacterium has been further modified to overexpress a gene encoding cysteine synthase.

It is another aspect of the invention to provide the method as described above, wherein said gene encoding cysteine synthase is overexpressed by increasing the copy number of the gene, by modifying an expression regulatory region of the gene, or a combination thereof, so that the expression of said gene encoding cysteine synthase is enhanced as compared with a non-modified bacterium.

It is another aspect of the invention to provide the method as described above, wherein said gene encoding cysteine synthase is a cysMgene.

It is another aspect of the invention to provide the method as described above, wherein said bacterium has been modified further to comprise a metA gene encoding a MetA protein, wherein the amino acid sequence of the MetA protein has the amino acid substitution R34C.

It is another aspect of the invention to provide the method as described above, wherein said bacterium has been further modified to attenuate expression of a metJ gene.

It is another aspect of the invention to provide the method as described above, wherein said metJ gene is deleted.

Still other objects, features, and attendant advantages of the present invention will become apparent to those skilled in the art from a reading of the following detailed description of embodiments constructed in accordance therewith.

### DETAILED DESCRIPTION

1. Bacterium

The bacterium as described herein is an L-methionine-producing bacterium that has been modified to overexpress a 1st gene and a 2nd gene as described herein. The bacterium as described herein can be used in the method as described herein. Hence, the explanations given hereinafter to the bacterium can be applied similarly to any bacterium that can be used interchangeably or equivalently in the method as described herein.

Any L-methionine-producing bacterium that has been modified to overexpress the 1st and 2nd genes can be used in the method as described herein. For example, an L-methionine-producing bacterium can be used in the method as described herein, provided that the bacterium has been modified to overexpress the 1st and 2nd genes, so that the production of L-methionine by the bacterium is enhanced as compared with a non-modified bacterium. The bacterium thus modified, for example, may be able to cause accumulation in a medium and/or cells of the bacterium of a higher amount of L-methionine as compared with a non-modified bacterium.

The phrase “an L-methionine-producing bacterium” may be used interchangeably or equivalently to the phrase “a bacterium that is able to produce L-methionine” or the phrase “a bacterium having an ability to produce L-methionine”.

The phrase “an L-methionine-producing bacterium” can mean a bacterium which has an ability to produce, excrete or secrete, and/or cause accumulation of L-methionine in a culture medium and/or cells of the bacterium when the bacterium is cultured in the medium.

The phrase “an L-methionine-producing bacterium” can also mean a bacterium which has an ability to produce, excrete or secrete, and/or cause accumulation of L-methionine in a culture medium in an amount larger than a non-modified bacterium. The phrase “a non-modified bacterium” may be used interchangeably or equivalently to the phrase “a non-modified strain”. The phrase “a non-modified strain” can mean a control strain that has not been modified to overexpress the 1st and 2nd genes, and can particularly mean a control strain that has not been modified to overexpress either the 1st or 2nd gene. Examples of the non-modified strain can include a wild-type or parental strain such as, for example, Pantoea ananatis (P. ananatis) AJ13355 strain. The phrase “an L-methionine-producing bacterium” can also mean a bacterium that is able to cause accumulation in the medium of an amount, for example, not less than 0.1 g/L, not less than 0.5 g/L, or not less than 1.0 g/L of L-methionine. The phrase “an L-methionine-producing bacterium” can also mean a bacterium which has an ability to produce, excrete or secrete, and/or cause accumulation of L-methionine in a culture medium in an amount larger than a non-modified bacterium, and is able to cause accumulation in the medium of an amount, for example, not less than 0.1 g/L, not less than 0.5 g/L, or not less than 1.0 g/L of L-methionine.

The bacterium may inherently have the ability to produce L-methionine or may be modified to have an ability to produce L-methionine. Such modification can be attained by using, for example, a mutation method or DNA recombination techniques. The bacterium can be obtained by overexpressing the 1st and 2nd genes in a bacterium that inherently has the ability to produce L-methionine, or in a bacterium that has been already imparted with the ability to produce L-methionine. Alternatively, the bacterium can be obtained by imparting the ability to produce L-methionine to a bacterium already modified to overexpress the 1st and 2nd genes. Alternatively, the bacterium may have been imparted with the ability to produce L-methionine by being modified to overexpress the 1st and 2nd genes. The bacterium as described herein can be obtained, specifically, for example, by modifying a bacterial strain described hereinafter.

The phrase “an ability to produce L-methionine” can mean the ability of a bacterium to produce, excrete or secrete, and/or cause accumulation of L-methionine in a culture medium and/or cells of the bacterium when the bacterium is cultured in the medium. The phrase “an ability to produce L-methionine” can specifically mean the ability of a bacterium to produce, excrete or secrete, and/or cause accumulation of L-methionine in a culture medium and/or cells of the bacterium to such a level that L-methionine can be collected from the culture medium and/or the cells when the bacterium is cultured in the medium.

The phrase “cultured” with reference to a bacterium which can be used in the method as described herein may be used interchangeably or equivalently to the phrase “cultivated”, or the like, that are well-known to the persons skilled in the art.

The bacterium can produce L-methionine either alone or as a mixture of L-methionine and one or more kinds of amino acids that are different from the L-methionine such as, for example, amino acids in L-form (also referred to as L-amino acids). Furthermore, the bacterium can produce L-methionine either alone or as a mixture of L-methionine and one or more other kinds of organic acids such as, for example, carboxylic acids. Examples of L-amino acids can include, but are not limited to, L-alanine, L-arginine, L-asparagine, L-aspartic acid, L-citrulline, L-cysteine, L-glutamic acid, L-glutamine, glycine, L-histidine, L-isoleucine, L-leucine, L-lysine, L-methionine, L-ornithine, L-phenylalanine, L-proline, L-serine, L-threonine, L-tryptophan, L-tyrosine, and L-valine. Examples of carboxylic acids can include, but are not limited to, formic acid, acetic acid, citric acid, butyric acid, lactic acid, and propionic acid, and derivatives thereof.

The phrases “L-methionine”, “an L-amino acid”, and “a carboxylic acid” can refer not only to L-methionine, an L-amino acid, and a carboxylic acid in a free form, but may also include a derivative form thereof, such as a salt, a hydrate, an adduct, or a combination of these. An adduct can be a compound formed by L-methionine, the L-amino acid, or the carboxylic acid in combination with another organic or inorganic compound. Hence, the phrases “L-methionine”, “an L-amino acid”, and “a carboxylic acid” can mean, for example, L-methionine, an L-amino acid, and a carboxylic acid in a free form, a derivative form, or a mixture of these. The phrases “L-methionine”, “an L-amino acid”, and “a carboxylic acid” can particularly mean, for example, L-methionine, an L-amino acid, and a carboxylic acid in a free form, a salt thereof, or a mixture of them. The phrases “L-methionine”, “an L-amino acid”, and “a carboxylic acid” can include, for example, sodium, potassium, ammonium, mono-, di- and trihydrate, mono- and dichlorhydrate, and so forth salts of them. Unless otherwise stated, the phrases “L-methionine”, “an L-amino acid”, and “a carboxylic acid” without referring to hydration, such as the phrases “L-methionine, an L-amino acid, or a carboxylic acid in a free form” and “a salt of L-methionine, an L-amino acid, or a carboxylic acid”, each can include both an anhydrate and a hydrate thereof.

The bacterium that can be used in a method as described herein or can be modified to obtain the bacterium as described herein can be, for example, a bacterium belonging to the family Enterobacteriaceae. Examples of the bacteria belonging to the family Enterobacteriaceae include bacteria belonging to the genera Enterobacter, Erwinia, Escherichia, Klebsiella, Morganella, Pantoea, Photorhabdus, Providencia, Salmonella, Yersinia, and so forth. Such bacteria can have the ability to produce L-methionine. Specifically, bacteria classified into the family Enterobacteriaceae according to the taxonomy used in the NCBI (National Center for Biotechnology Information) database (ncbi.nlm.nih.gov/Taxonomy/Browser/wwwtax.cgi?id=543) can be used. Particular examples of the bacteria belonging to the family Enterobacteriaceae include bacteria belonging to the genera Escherichia, Enterobacter, and Pantoea.

Escherichia bacteria are not particularly limited, and examples thereof include those described in the work of Neidhardt et al. (Bachmann, B. J., Derivations and genotypes of some mutant derivatives of E. coli K-12, p. 2460-2488. In F. C. Neidhardt et al. (ed.), E. coli and Salmonella: cellular and molecular biology, 2nd ed. ASM Press, Washington, D.C., 1996). The species Escherichia coli (E. coli) is a particular example of Escherichia bacteria. Specific examples of E. coli include E. coli K-12 strain, which is a prototype wild-type strain, such as E. coli W3110 (ATCC 27325), E. coli MG1655 (ATCC 47076), and so forth.

Examples of the Enterobacter bacteria include Enterobacter agglomerans, Enterobacter aerogenes, and so forth. As the bacterium belonging to the genus Pantoea, any bacterium that is classified into the genus Pantoea according to the taxonomy used in the NCBI (National Center for Biotechnology Information) database (ncbi.nlm.nih.gov/Taxonomy/Browser/wwwtax.cgi?mode=Tree&id=53335&1v1=3&keep=l&s rchmode=1&unlock) can be used. Examples of the Pantoea bacteria include Pantoea ananatis (P. ananatis), and so forth. Some strains of Enterobacter agglomerans were recently reclassified into Pantoea agglomerans, Pantoea ananatis or Pantoea stewartii on the basis of nucleotide sequence analysis of 16S rRNA, etc. A bacterium belonging to either genus Enterobacter or Pantoea may be used so long as it is a bacterium classified into the family Enterobacteriaceae. Specific examples of P. ananatis include Pantoea ananatis AJ13355 strain (FERM BP-6614), AJ13356 strain (FERM BP-6615), AJ13601 strain (FERM BP-7207), and derivatives thereof. These strains were identified as Enterobacter agglomerans when they were isolated, and deposited as Enterobacter agglomerans. However, they were recently reclassified as Pantoea ananatis on the basis of nucleotide sequencing of 16S rRNA and so forth as described above.

These strains are available from, for example, the American Type Culture Collection (ATCC; Address: 10801 University Boulevard, Manassas, Va. 20110, United States of America). That is, registration numbers are assigned to the respective strains, and the strains can be ordered by using these registration numbers (refer to lgcstandards-atcc.org). The registration numbers of the strains are listed in the catalogue of the American Type Culture Collection. These strains can also be obtained from, for example, the depositories at which the respective strains were deposited.

L-Methionine-Producing Bacteria

Examples of L-methionine-producing bacteria and parental strains for deriving them include L-threonine auxotrophic strains and mutant strains resistant to norleucine (Japanese Patent Laid-open (Kokai) No. 2000-139471). Examples of L-methionine-producing bacteria and parental strains for deriving them also include a strain containing a mutant homoserine transsuccinylase resistant to feedback inhibition by L-methionine (Japanese Patent Laid-open (Kokai) No. 2000-139471, US2009-0029424A). Since L-methionine is biosynthesized via L-cysteine as an intermediate, L-methionine-producing ability can also be improved by improving L-cysteine-producing ability (Japanese Patent Laid-open (Kokai) No. 2000-139471, U52008-0311632A).

Examples of L-methionine-producing bacteria of the genus Pantoea and parent strains thereof that can be used to derive L-methionine-producing bacteria can include, but are not limited to, P. ananatis strain AJ13355 (FERM BP-6614), P. ananatis SC17 strain (FERM BP-11091), and P. ananatis SC17(0) strain (VKPM B-9246). The AJ13355 strain is a strain isolated from soil in Iwata-shi (Shizuoka, Japan) as a bacterium that is able to grow at acidic pH and showing resistance to high concentrations of glutamic acid (U.S. Pat. No. 7,319,025 B2; Katashkina J. I. et al., Use of the λRed-recombineering method for genetic engineering of Pantoea ananatis, BMC Mol. Biol., 2009, 10:34). The SC17 strain is a strain selected as a low phlegm-producing mutant strain from the AJ13355 strain (U.S. Pat. No. 6,596,517). The SC17(0) strain was constructed as a strain resistant to the Red gene products for performing gene disruption in P. ananatis (WO2008075483). The SC17 strain was deposited at the independent administrative agency, National Institute of Advanced Industrial Science and Technology, International Patent Organism Depository (currently independent administrative agency, National Institute of Technology and Evaluation, International Patent Organism Depositary, #120, 2-5-8 Kazusakamatari, Kisarazu-shi, Chiba-ken, 292-0818, Japan) on Feb. 4, 2009, and assigned an accession number of FERM BP-11091. The strain SC17(0) was deposited in the Russian National Collection of Industrial Microorganisms (VKPM; FGUP GosNII Genetika, Russian Federation, 117545 Moscow, 1st Dorozhny proezd, 1) on Sep. 21, 2005 under the accession number VKPM B-9246.

Examples of L-methionine-producing bacteria of the genus Escherichia and parent strains thereof that can be used to derive L-methionine-producing bacteria can include, but are not limited to, E. coli strain that is deficient in repressor of L-methionine biosynthesis system (MetJ) and has increased activity of intracellular homoserine transsuccinylase (MetA) (U.S. Pat. No. 7,611,873 B1), E. coli strain in which activity of cobalamin-independent methionine synthase (MetE) is suppressed and activity of cobalamin-dependent methionine synthase (MetH) is increased (EP2861726 B1), E. coli strain that has an ability to produce L-threonine and is transformed with vector(s) expressing threonine dehydratase (tdcB, ilvA) and, at least, O-succinylhomoserine lyase (metB), cystathionine beta-lyase (metC), 5,10-methylenetetrahydrofolate reductase (metF) and serine hydroxymethyltransferase (glyA) (U.S. Pat. No. 7,790,424 B2), E. coli strain in which activity of transhydrogenase (pntAB) is enhanced (EP2633037 B1), and so forth. Specific examples of L-methionine-producing bacteria of the genus Escherichia and parent strains thereof that can be used to derive L-methionine-producing bacteria can include, for example, E. coli AJ11539 (NRRL B-12399), E. coli AJ11540 (NRRL B-12400), E. coli AJ11541 (NRRL B-12401), E. coli AJ11542 (NRRL B-12402, British Patent No. 2075055), the E. coli 218 strain (VKPM B-8125, Russian Patent No. 2209248) and the 73 strain (VKPM B-8126, Russian Patent No. 2215782), which are resistant to norleucine, which is an analogue of L-methionine, and E. coli AJ13425 (FERM P-16808, Japanese Patent Laid-open (Kokai) No. 2000-139471). The AJ13425 strain is an L-threonine auxotrophic strain derived from the E. coli W3110, in which the methionine repressor is deleted, the intracellular S-adenosylmethionine synthetase activity is attenuated, and the intracellular homoserine transsuccinylase activity, cystathionine γ-synthase activity, and aspartokinase-homoserine dehydrogenase II activity are enhanced.

The bacterium as described herein can have, for example, one or more properties, such as modifications, selected from those possessed by the L-methionine-producing bacteria exemplified above.

The genes and proteins used for breeding L-methionine-producing bacteria may have, for example, known nucleotide sequences and amino acid sequences of the genes and proteins exemplified above, respectively. Also, the genes and proteins used for breeding L-methionine-producing bacteria may be variants of the genes and proteins exemplified above, such as genes and proteins having such known nucleotide sequences and amino acid sequences, respectively, so long as the original function thereof, such as respective enzymatic activities in cases of proteins, is maintained. As for variants of genes and proteins, the descriptions concerning variants of a 1st gene and a 1st protein described herein can be applied similarly.

The bacterium as described herein has been modified to overexpress a 1st gene and a 2nd gene as described herein.

The phrase “a 1st gene” can refer to a gene, such as DNA, having the nucleotide sequence shown in SEQ ID NO: 1 or a variant sequence thereof. A protein encoded by the 1st gene can also be referred to as “a 1st protein”. That is, the phrase “a 1st gene” can also refer to a gene encoding a 1st protein.

The phrase “a 2nd gene” can refer to a gene, such as DNA, having the nucleotide sequence shown in SEQ ID NO: 3 or a variant sequence thereof. A protein encoded by the 2nd gene can also be referred to as “a 2nd protein”. That is, the phrase “a 2nd gene” can also refer to a gene encoding a 2nd protein.

A gene having the nucleotide sequence shown in SEQ ID NO: 1 native to P. ananatis has the accession No. PAJ_RS05335 in the BioCyc database (biocyc.org). The gene encodes a protein having the amino acid sequence shown in SEQ ID NO: 2, which can be a hypothetical protein the activity or function of which is not known. That is, examples of the 1st protein include a protein having the amino acid sequence of SEQ ID NO: 2.

A gene having the nucleotide sequence shown in SEQ ID NO: 3 native to P. ananatis has the accession No. PAJ_RS05340 in the BioCyc database. The gene encodes a protein having the amino acid sequence shown in SEQ ID NO: 4, which can be a putative 5-methyltetrahydropteroyltriglutamate-homocysteine methyltransferase that causes catalysis of the following reaction: L-homocysteine+5-methyltetrahydropteroyltri-L-glutamate <-> tetrahydropteroyl-L-glutamate+L-methionine (Enzyme Commission (EC) No.: 2.1.1.14). That is, examples of the 2nd protein include a protein having the amino acid sequence of SEQ ID NO: 4.

The phrase “a gene or protein has a nucleotide or amino acid sequence” can mean that a gene or protein includes the nucleotide or amino acid sequence unless otherwise stated, and can also include cases where a gene or protein includes only the nucleotide or amino acid sequence.

Hereinafter, variants of the 1st gene and variants of the 1st protein, specifically variants of a gene having the nucleotide sequence shown in SEQ ID NO: 1 and variants of a protein having the amino acid sequence shown in SEQ ID NO: 2, will be mainly described. The below descriptions concerning such variants of the gene and protein can also be applied similarly to any gene and protein, including the 2nd gene and the 2nd protein and any other gene and protein.

There may be differences in DNA sequences between the genera, species or strains of bacteria. Therefore, the 1st gene is not limited to the gene having the nucleotide sequence shown in SEQ ID NO: 1, but may include genes, such as DNAs, having a variant nucleotide sequence of SEQ ID NO: 1, and having the function of the 1st gene. Similarly, the 1st protein is not limited to the protein having the amino acid sequence shown in SEQ ID NO: 2, but may include proteins having a variant amino acid sequence of SEQ ID NO: 2 and having the function of the 1st protein. Examples of such variant nucleotide sequences or variant amino acid sequences may include homologues of, and artificially modified sequences of the 1st gene and the 1st protein exemplified above.

The phrase “a gene has the function of the 1st gene” can mean that the gene has a property that if the gene is overexpressed in the bacterium, the amount of L-methionine produced by the bacterium is increased as compared with that observed for a non-modified strain. It can be sufficient that the amount of L-methionine produced by the bacterium is increased when the 1st gene is overexpressed in combination with an appropriate 2nd gene. Hence, the phrase “a gene has the function of the 1st gene” can specifically mean that a gene has a property that if the gene is overexpressed in combination with a 2nd gene in the bacterium, the amount of L-methionine produced by the bacterium is increased as compared with that observed for a non-modified strain. The phrase “a gene has the function of the 1st gene” can also mean that a gene encodes a protein having the function of the 1st protein. The phrase “a protein has the function of the 1st protein” can mean that a protein has the function of the protein having the amino acid sequence shown in SEQ ID NO: 2. Examples of the function of a protein can include the activity of the protein. The phrase “a protein has the function of the 1st protein” can also mean that the protein has a property that if the amount of protein is increased in the bacterium, the amount of L-methionine produced by the bacterium is increased as compared with that observed for a non-modified strain. It can be sufficient that the amount of L-methionine produced by the bacterium is increased when the amount of the 1st protein is increased in combination with an appropriate 2nd protein. Hence, the phrase “a protein has the function of the 1st protein” can specifically mean that the protein has a property that if the amount of protein is increased in combination with a 2nd protein in the bacterium, the amount of L-methionine produced by the bacterium is increased as compared with that observed for a non-modified strain.

The 2nd gene is not limited to the gene having the nucleotide sequence shown in SEQ ID NO: 3, but may include genes, such as DNAs, having a variant nucleotide sequence of SEQ ID NO: 3, and having the function of the 2nd gene. Similarly, the 2nd protein is not limited to the protein having the amino acid sequence shown in SEQ ID NO: 4, but may include proteins having a variant amino acid sequence of SEQ ID NO: 4 and having the function of the 2nd protein. Examples of such variant nucleotide sequences or variant amino acid sequences may include homologues of, and artificially modified sequences of the 2nd gene and the 2nd protein exemplified above.

The phrase “a gene has the function of the 2nd gene” can mean that the gene has a property that if the gene is overexpressed in the bacterium, the amount of L-methionine produced by the bacterium is increased as compared with that observed for a non-modified strain. It can be sufficient that the amount of L-methionine produced by the bacterium is increased when the 2nd gene is overexpressed in combination with an appropriate 1st gene. Hence, the phrase “a gene has the function of the 2nd gene” can specifically mean that the gene has a property that if the gene is overexpressed in combination with the 1st gene in the bacterium, the amount of L-methionine produced by the bacterium is increased as compared with that observed for a non-modified strain. The phrase “a gene has the function of the 2nd gene” can also mean that the gene encodes a protein having the function of the 2nd protein. The phrase “a protein has the function of the 2nd protein” can mean that the protein has the function of the protein having the amino acid sequence shown in SEQ ID NO: 4. Examples of the function of a protein can include the activity of the protein. The phrase “a protein has the function of the 2nd protein” can also mean that the protein has a property that if the amount of protein is increased in the bacterium, the amount of L-methionine produced by the bacterium is increased as compared with that observed for a non-modified strain. It can be sufficient that the amount of L-methionine produced by the bacterium is increased when the amount of the 2nd protein is increased in combination with an appropriate 1st protein. Hence, the phrase “a protein has the function of the 2nd protein” can specifically mean that a protein has a property that if the amount of protein is increased in combination with a 1st protein in the bacterium, the amount of L-methionine produced by the bacterium is increased as compared with that observed for a non-modified strain.

The phrase “a variant nucleotide sequence” in reference to the 1st gene can mean a nucleotide sequence which encodes the 1st protein, such as the protein having the amino acid sequence shown in SEQ ID NO: 2, using any synonymous amino acid codons according to the standard genetic code table (see, for example, Lewin B., “Genes VIII”, 2004, Pearson Education, Inc., Upper Saddle River, N.J. 07458). Therefore, the 1st gene can be a gene having a variant nucleotide sequence of SEQ ID NO: 1 due to the degeneracy of the genetic code.

The phrase “a variant nucleotide sequence” in reference to the 1st gene can also mean a nucleotide sequence that is able to hybridize under stringent conditions with the nucleotide sequence complementary to the sequence shown in SEQ ID NO: 1. The phrase “stringent conditions” can include those conditions under which a specific hybrid, for example, a hybrid having homology, defined as the parameter “identity” when using the computer program blastn, not less than 85%, not less than 90%, not less than 91%, not less than 92%, not less than 93%, not less than 94%, not less than 95%, not less than 96%, not less than 97%, not less than 98%, or not less than 99% is formed, and a non-specific hybrid, for example, a hybrid having homology lower than the above is not formed. For example, stringent conditions can be exemplified by washing one time or more, or in another example, two or three times, at a salt concentration of 1×SSC (standard sodium citrate or standard sodium chloride), 0.1% SDS (sodium dodecyl sulphate) at 60° C., 0.1×SSC, 0.1% SDS at 60° C., or 0.1×SSC, 0.1% SDS at 65° C. Duration of washing can depend on the type of membrane used for the blotting and, as a rule, should be what is recommended by the manufacturer. For example, the recommended duration of washing for the Amersham Hybond™-N+ positively charged nylon membrane (GE Healthcare) under stringent conditions is 15 minutes. The washing step can be performed 2 to 3 times. As the probe, a part of the sequence complementary to the sequence shown in SEQ ID NO: 1 may also be used. Such a probe can be produced by PCR (polymerase chain reaction; refer to White T. J. et al., The polymerase chain reaction, Trends Genet., 1989, 5:185-189) using oligonucleotides as primers prepared on the basis of the sequence shown in SEQ ID NO: 1 and a DNA fragment containing the nucleotide sequence as a template. The length of the probe is recommended to be >50 bp; it can be suitably selected depending on the hybridization conditions, and is usually 100 bp to 1 kbp. For example, when a DNA fragment having a length of about 300 bp is used as the probe, the washing conditions after the hybridization can be, for example, 2×SSC, 0.1% SDS at 50° C., 60° C. or 65° C.

The phrase “a variant nucleotide sequence” in reference to the 1st gene can also mean a nucleotide sequence that encodes a variant protein of the 1st protein.

The phrase “a variant protein” in reference to the 1st protein can mean a protein which has a variant amino acid sequence of SEQ ID NO: 2.

The phrase “a variant protein” in reference to the 1st protein can specifically mean a protein which has one or more mutations in the sequence as compared with the amino acid sequence shown in SEQ ID NO: 2, whether they are substitutions, deletions, insertions, and/or additions of one or several amino acid residues, but which still maintains the function of the 1st protein, such as the function of the protein having the amino acid sequence shown in SEQ ID NO: 2, or in which the three-dimensional structure is not significantly changed relative to the non-modified protein such as, for example, the protein having the amino acid sequence shown in SEQ ID NO: 2. The number of changes in the variant protein depends on the position of amino acid residue(s) in the three-dimensional structure of the protein or the type of amino acid residue(s). It can be, but is not strictly limited to, 1 to 50, in another example 1 to 40, in another example 1 to 30, in another example 1 to 20 in another example 1 to 20, in another example 1 to 15, in another example 1 to 10, and in another example 1 to 5, in SEQ ID NO: 2. This is possible because amino acids can have high homology to one another, so that the function of a protein is not affected by a change between such amino acids, or the three-dimensional structure of a protein is not significantly changed relative to the corresponding non-modified protein by a change between such amino acids. Therefore, the variant protein may be a protein having an amino acid sequence having a homology, defined as the parameter “identity” when using the computer program blastp, not less than 85%, not less than 90%, not less than 91%, not less than 92%, not less than 93%, not less than 94%, not less than 95%, not less than 96%, not less than 97%, not less than 98%, or not less than 99% with respect to the entire amino acid sequence shown in SEQ ID NO: 2 as long as the function of the protein is maintained, or the three-dimensional structure of the protein is not significantly changed relative to the non-modified protein such as, for example, the protein having the amino acid sequence shown in SEQ ID NO: 2. In this specification, “homology” may mean “identity”, that is the identity of amino acid residues. The sequence identity between two sequences is calculated as the ratio of residues matching in the two sequences when aligning the two sequences so as to achieve a maximum alignment with each other.

The exemplary substitution, deletion, insertion, and/or addition of one or several amino acid residues can be a conservative mutation(s). The representative conservative mutation can be a conservative substitution. The conservative substitution can be, but is not limited to, a substitution, wherein substitution takes place mutually among Phe, Trp and Tyr, if the substitution site is an aromatic amino acid; among Ala, Leu, Ile and Val, if the substitution site is a hydrophobic amino acid; between Glu, Asp, Gln, Asn, Ser, His and Thr, if the substitution site is a hydrophilic amino acid; between Gln and Asn, if the substitution site is a polar amino acid; among Lys, Arg and His, if the substitution site is a basic amino acid; between Asp and Glu, if the substitution site is an acidic amino acid; and between Ser and Thr, if the substitution site is an amino acid having hydroxyl group. Examples of conservative substitutions include substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. In addition, such substitution, deletion, insertion, addition or the like of amino acid residues as mentioned above includes a naturally occurring mutation due to an individual difference of an organism to which the amino acid sequence is native.

The exemplary substitution, deletion, insertion, and/or addition of one or several amino acid residues can also be a non-conservative mutation(s) provided that the mutation(s) is/are compensated by one or more secondary mutation(s) in a different position(s) of the amino acid sequence so that the activity or function of the variant protein is maintained, or the three-dimensional structure of the protein is not significantly changed relative to the non-modified protein such as, for example, the protein having the amino acid sequence shown in SEQ ID NO: 2.

Since the nucleotide sequence of the 1st gene native to P. ananatis (SEQ ID NO: 1) and the amino acid sequence of the 1st protein encoded by that gene (SEQ ID NO: 2) have already been elucidated (see above), the 1st gene native to P. ananatis or a variant nucleotide sequence thereof can be obtained by cloning from P. ananatis by PCR (polymerase chain reaction; refer to White T. J. et al., The polymerase chain reaction, Trends Genet., 1989, 5:185-189) utilizing DNA of P. ananatis and primers prepared based on the nucleotide sequence shown in SEQ ID NO: 1; or a mutagenesis method of treating a DNA containing the 1st gene native to P. ananatis in vitro, for example, with hydroxylamine, or a mutagenesis method of treating P. ananatis harboring the 1st gene with ultraviolet (UV) irradiation or a mutating agent such as N-methyl-N′-nitro-N-nitrosoguanidine (NTG) and nitrous acid usually used for the such treatment; or chemical synthesis as a full-length gene structure. The 1st gene native to any other organism or a variant nucleotide sequence thereof can be obtained in a similar manner.

The calculation of a percent identity of a polypeptide can be carried out using the algorithm blastp. More specifically, the calculation of a percent identity of a polypeptide can be carried out using the algorithm blastp in the default settings of Scoring Parameters (Matrix: BLOSUM62; Gap Costs: Existence=11 Extension=1; Compositional Adjustments: Conditional compositional score matrix adjustment) provided by National Center for Biotechnology Information (NCBI). The calculation of a percent identity of a polynucleotide can be carried out using the algorithm blastn. More specifically, the calculation of a percent identity of a polynucleotide can be carried out using the algorithm blastn in the default settings of Scoring Parameters (Match/Mismatch Scores=1, −2; Gap Costs=Linear) provided by NCBI.

The phrase “a bacterium has been modified to overexpress a 1st gene” can mean that the bacterium has been modified in such a way that in the modified bacterium, the total amount and/or the total activity of the corresponding gene product of the 1st gene, specifically the total amount and/or the total activity of the 1st protein or the expression level (that is, expression amount) of the 1st gene, is increased as compared with (that is, higher than), that observed for a non-modified strain. The phrase “a non-modified strain” can refer to a bacterial strain that can serve as a reference for the above comparison. The phrase “a non-modified strain” can be used interchangeably or equivalently to the phrases “a non-modified bacterium” and “a non-modified bacterial strain”. Examples of a non-modified strain can include, for example, a wild-type or parental strain. Specific examples of a non-modified strain can include a wild-type strain of a bacterium belonging to the family Enterobacteriaceae such as, for example, the P. ananatis AJ13355 strain (FERM BP-6614), E. coli W3110 strain (ATCC 27325), E. coli MG1655 strain (ATCC 47076), and so forth.

The total amount and/or the total enzymatic activity of the corresponding gene product of the 1st gene, specifically the total amount and/or the total activity of a 1st protein, can be increased by, for example, increasing (that is, enhancing) the expression level of said 1st gene, or increasing the activity per molecule (may be referred to as a specific activity) of the 1st protein encoded by said 1st gene, as compared with a non-modified strain, for example, a wild-type or parental strain. An increase in the total amount or the total activity of a protein can be measured as, for example, an increase in the amount or activity of the protein per cell, which may be an average amount or activity of the protein per cell. The bacterium can be modified so that the amount and/or the activity of the corresponding protein per cell is increased to 150% or more, 200% or more, or 300% or more, of the amount and/or the activity of a non-modified strain.

The phrase “a bacterium has been modified to overexpress a 1st gene” can also mean that the bacterium has been modified in such a way that in the modified bacterium, the expression level (that is, expression amount) of a 1st gene is enhanced or increased as compared with a non-modified strain, for example, a wild-type or parental strain. Therefore, the phrase “a gene is overexpressed” can be used interchangeably or equivalently to the phrase “the expression of a gene is enhanced or increased” or the phrase “the expression level of a gene is enhanced or increased”. Furthermore, the phrase “a bacterium has been modified to overexpress a 1st gene” can also mean that the expression level of a 1st gene in the modified bacterium is higher than that observed for a non-modified strain. An increase in the expression level of a gene can be measured as, for example, an increase in the expression level of the gene per cell, which may be an average expression level of the gene per cell. The phrase “the expression level of a gene” or “the expression amount of a gene” can mean, for example, the amount of an expression product of a gene, such as the amount of mRNA of the gene or the amount of the protein encoded by the gene. The bacterium may be modified so that the expression level of the 1st gene per cell is increased to, for example, 150% or more, 200% or more, or 300% or more, of the expression level of the 1st gene in a non-modified strain.

The protein concentration can be determined by the Bradford protein assay, the method of Lowry using bovine serum albumin (BSA) as a standard and a Coomassie dye, or a Western blot analysis (Bradford M. M., Anal. Biochem., 1976, 72:248-254; Lowry O. H. et al., J. Biol. Chem., 1951, 193:265-275; Belogurov G. A. et al., 2002).

The aforementioned descriptions concerning overexpression of the 1st gene can also be applied similarly to overexpression of any gene, including the 2nd gene and any other gene.

Examples of methods which can be used to overexpress a gene such as the 1st and 2nd genes can include, but are not limited to, a method of increasing the copy number of the gene, such as the copy number of the gene in the chromosome of the bacterium and/or in the autonomously replicating vector, such as a plasmid, harbored by the bacterium. The copy number of a gene can be increased by, for example, introducing the gene into the chromosome of the bacterium and/or introducing an autonomously replicating vector containing the gene into the bacterium. Such increasing of the copy number of a gene can be carried out according to genetic engineering methods known to the one of ordinary skill in the art.

Examples of the vectors that can be used for a bacterium belonging to the family Enterobacteriaceae can include, but are not limited to, broad-host-range plasmids such as pMW118/119, pBR322, pUC19, pAH162, RSF1010, RP4, and the like. A gene can also be introduced into the chromosomal DNA of a bacterium by, for example, homologous recombination, Mu-driven integration, or the like. Only one copy, or two or more copies of a gene may be introduced. For example, homologous recombination can be carried out using a nucleotide sequence, multiple copies of which exist in the chromosomal DNA as a target, to introduce multiple copies of a gene into the chromosomal DNA. Examples of a nucleotide sequence, multiple copies of which exist in the chromosomal DNA, can include, but are not limited to, repetitive DNA, and inverted repeats present at the end of a transposable element. In addition, it is possible to incorporate a gene into a transposon and allow it to be transferred to introduce multiple copies of the gene into the chromosomal DNA. A method for intrachromosomal amplification can be used to introduce multiple copies of a gene into the chromosomal DNA. By using Mu-driven transposition, more than 3 copies of the gene can be introduced into the chromosomal DNA of recipient strain in one step (Akhverdyan V. Z. et al., Biotechnol. (Russian), 2007, 3:3-20).

A gene to be introduced into the bacterium as described herein can be ligated downstream from a promoter. The promoter is not particularly limited so long as the promoter that can function in the host bacterium is chosen, and it may be a promoter native to the host bacterium, or it may be a heterologous promoter. The phrase “a promoter that can function in a host bacterium” can refer to a promoter that possesses promoter activity in a host bacterium. Specific examples of a promoter that can function in a bacterium belonging to the family Enterobacteriaceae can include, but are not limited to, potent promoters exemplified below.

Examples of methods which can be used to overexpress a gene such as the 1st and 2nd genes also can include a method of increasing the expression level of the gene by modification of an expression regulatory region of the gene. Modification of an expression regulatory region of a gene can be employed in combination with an increase in the copy number of the gene. An expression regulatory region of a gene can be modified by, for example, replacing the native expression regulatory region of the gene with a native and/or modified foreign expression regulatory region. The phrase “an expression regulatory region” can be used interchangeably or equivalently to the phrase “an expression regulatory sequence”.

Expression regulatory regions can be exemplified by promoters, enhancers, operators, attenuators and termination signals, anti-termination signals, ribosome-binding sites (RBS) and other expression control elements, for example, regions to which repressors or activators bind and/or binding sites for transcriptional and translational regulatory proteins, for example, in the transcribed mRNA. Such regulatory regions are described, for example, in known documents (Sambrook J., Fritsch E. F. and Maniatis T., “Molecular Cloning: A Laboratory Manual”, 2nd ed., Cold Spring Harbor Laboratory Press (1989); Pfleger B. F. et al., Combinatorial engineering of intergenic regions in operons tunes expression of multiple genes, Nat. Biotechnol., 2006, 24:1027-1032; Mutalik V. K. et al., Precise and reliable gene expression via standard transcription and translation initiation elements, Nat. Methods, 2013, 10:354-360). Modifications of an expression regulatory region of a gene can be combined with increasing the copy number of the gene (see, for example, Akhverdyan V. Z. et al., Appl. Microbiol. Biotechnol., 2011, 91:857-871; Tyo K. E. J. et al., Nature Biotechnol., 2009, 27:760-765).

The exemplary promoters suitable for enhancing expression of a gene can be potent promoters. The phrase “a potent promoter” can refer to a promoter stronger than the native promoter of a gene. Examples of potent promoters that can function in a bacterium belonging to the family Enterobacteriaceae can include, but are not limited to, lac promoter, trp promoter, trc promoter, tac promoter, tet promoter, araBAD promoter, rpoH promoter, msrA promoter, Pm1 promoter (derived from the genus Bifidobacterium), and PR or the PL promoters of phage. As a potent promoter, a highly active variant of an existing promoter may also be obtained by using various reporter genes. For example, by making the −35 and −10 regions in a promoter region closer to a consensus sequence, the strength of the promoter can be enhanced (WO0018935 A1). The strength of a promoter can be defined by the frequency of initiation acts of RNA synthesis. Examples of the method for evaluating the strength of a promoter and examples of strong promoters are described in the paper of Goldstein M. A. et al. (Prokaryotic promoters in biotechnology, Biotechnol. Annu. Rev., 1995, 1:105-128) and so forth. Potent promoters providing a high level of gene expression in a bacterium such as, for example, a bacterium belonging to the family Enterobacteriaceae can be used. Alternatively, the effect of a promoter can be enhanced by, for example, introducing a mutation into the promoter region of the gene to obtain a stronger promoter function, thus resulting in the increased transcription level of the gene located downstream of the promoter. Furthermore, it is known that substitution of several nucleotides in the Shine-Dalgarno (SD) sequence, and/or in the spacer between the SD sequence and the start codon, and/or a sequence immediately upstream and/or downstream from the start codon in the ribosome-binding site greatly affects the translation efficiency of mRNA. Hence, these portions can be examples of expression regulatory regions of a gene. For example, a 20-fold range in the expression levels was found, depending on the nature of the three nucleotides preceding the start codon (Gold L. et al., Annu. Rev. Microbiol., 1981, 35:365-403; Hui A. et al., EMBO J., 1984, 3:623-629).

Virtually, any method for gene overexpression may be used so long as the overexpression of the 1st and 2nd genes can be attained using that method. Therefore, the 1st and 2nd genes can be overexpressed using one method for gene overexpression, or the 1st and 2nd genes can be overexpressed using different methods for gene overexpression. For example, the 1st and 2nd genes can be overexpressed in, that is, introduced into, the bacterium in such a way that the genes are present on different nucleic acid molecules. Alternatively, the 1st and 2nd genes can be overexpressed in, that is, introduced into, the bacterium in such a way that the genes are present on one nucleic acid molecule. For example, the 1st and 2nd genes may be present on one expression vector or on the chromosome. Alternatively, the 1st and 2nd genes may be present individually on two different expression vectors. Also, alternatively, one of the 1st and 2nd genes may be present on an expression vector and the other one may be present on the chromosome. The same shall apply to any combination of genes.

A method for the overexpression of a gene in a bacterium can be a method of introducing a nucleic acid (DNA) having the gene into the bacterium. Examples of methods for introducing a nucleic acid such as, for example, a gene, a vector, and the like, into a bacterium can include, but are not limited to, genetic engineering methods known to the person of ordinary skill in the art. For example, known methods for introducing a nucleic acid into a bacterium belonging to the family Enterobacteriaceae can be used.

In the bacterium as described herein, the 1st and 2nd genes each can be present on a vector that autonomously replicates outside of the chromosome such as a plasmid, or may be incorporated into the chromosome, or a combination of these. In addition, as described above, to construct the bacterium as described herein, introduction of the 1st and 2nd genes and impartation or enhancement of the ability to produce L-methionine can be performed in any order.

When two or more genes, such as the 1st and 2nd genes and other genes, are overexpressed, those two or more genes can be overexpressed using one method for gene overexpression, or those genes can be overexpressed using different methods for gene overexpression. Furthermore, those two or more genes can be overexpressed, for example, one by one or simultaneously.

Two or more genes, such as the 1st and 2nd genes and other genes, may be organized in an operon structure. Therefore, a method that can be used to enhance gene expression can also be applied to increase the expression level of the operon having two or more genes. For example, modification of expression regulatory region(s) of an operon or introducing native and/or modified foreign expression regulatory region(s) into an operon may be used to enhance expression of two or more genes in the operon. An operon, for example, may include the 1st and 2nd genes, and may further include one or more other gene(s). In this method, the expression of two or more genes can be enhanced at the same time.

Methods as described herein for overexpression of a gene, such as the 1st and 2nd genes, can be applied similarly to overexpression of any gene.

The copy number of a gene or the presence or absence of a gene can be measured, for example, by restricting the chromosomal DNA followed by Southern blotting using a probe based on the gene sequence, fluorescence in situ hybridization (FISH), and the like. The level of gene expression can be determined by measuring the amount of mRNA transcribed from the gene using various well-known methods, including Northern blotting, quantitative RT-PCR, and the like. The amount of the protein encoded by the gene can be measured by known methods including SDS-PAGE followed by immunoblotting assay (Western blotting analysis), or mass spectrometry analysis of the protein samples, and the like.

Methods for manipulation with recombinant molecules of DNA and molecular cloning such as preparation of plasmid DNA, digestion, ligation and transformation of DNA, selection of an oligonucleotide as a primer, incorporation of mutations, and the like may be ordinary methods well-known to the persons skilled in the art. These methods are described, for example, in Sambrook J., Fritsch E. F. and Maniatis T., “Molecular Cloning: A Laboratory Manual”, 2^{nd} ed., Cold Spring Harbor Laboratory Press (1989) or Green M. R. and Sambrook J. R. “Molecular Cloning: A Laboratory Manual”, 4^{th} ed., Cold Spring Harbor Laboratory Press (2012); Bernard R. Glick, Jack J. Pasternak and Cheryl L. Patten, “Molecular Biotechnology: principles and applications of recombinant DNA”, 4^{th} ed., Washington, D.C., ASM Press (2009).

Any method for manipulation with recombinant DNA can be used including conventional methods such as, for example, transformation, transfection, infection, conjugation, and mobilization. Transformation, transfection, infection, conjugation, or mobilization of a bacterium with the DNA encoding a protein can impart to the bacterium the ability to synthesize the protein encoded by the DNA. Methods of transformation, transfection, infection, conjugation, and mobilization include any known method. For example, a method of treating recipient cells with calcium chloride so as to increase permeability of the cells of E. coli K-12 to DNA has been reported for efficient DNA transformation and transfection (Mandel M. and Higa A., Calcium-dependent bacteriophage DNA infection, J. Mol. Biol., 1970, 53:159-162). Methods of specialized and/or generalized transduction have been described (Morse M. L. et al., Transduction in Escherichia coli K-12, Genetics, 1956, 41(1):142-156; Miller J. H., Experiments in Molecular Genetics. Cold Spring Harbor, N.Y.: Cold Spring Harbor La. Press, 1972). Other methods for random and/or targeted integration of DNA into the host microorganism can be applied, for example, “λ Red-recombineering” (Katashkina J. I. et al., Use of the λ Red-recombineering method for genetic engineering of Pantoea ananatis, BMC Mol. Biol., 2009, 10:34), “Mu-driven integration/amplification” (Akhverdyan et al., Appl. Microbiol. Biotechnol., 2011, 91:857-871), “Red/ET-driven integration” or “λRed/ET-mediated integration” (Datsenko K. A. and Wanner B. L., Proc. Natl. Acad. Sci. USA 2000, 97(12):6640-45; Zhang Y., et al., Nature Genet., 1998, 20:123-128). Moreover, for multiple insertions of desired genes in addition to Mu-driven replicative transposition (Akhverdyan et al., Appl.Microbiol. Biotechnol., 2011, 91:857-871) and chemically inducible chromosomal evolution based on recA-dependent homologous recombination resulted in an amplification of desired genes (Tyo K. E. J. et al., Nature Biotechnol., 2009, 27:760-765), other methods can be used, which utilize different combinations of transposition, site-specific and/or homologous Red/ET-mediated recombinations, and/or P1-mediated generalized transduction (see, for example, Minaeva N. I. et al., BMC Biotechnology, 2008, 8:63; Koma D. et al., Appl. Microbiol. Biotechnol., 2012, 93(2):815-829).

The phrase “native to” in reference to a protein or a nucleic acid can mean that the protein or the nucleic acid is native to a particular organism such as, for example, a mammal, plant, insect, bacterium, or virus. That is, a protein or a nucleic acid native to a particular organism can mean the protein or the nucleic acid, respectively, that exists naturally in that organism. A protein or a nucleic acid native to a particular organism can be isolated from that organism and sequenced using means known to the one of ordinary skill in the art. Moreover, as the amino acid sequence or the nucleotide sequence of a protein or nucleic acid, respectively, isolated from an organism in which the protein or nucleic acid exists, can easy be determined, the phrase “native to” in reference to a protein or a nucleic acid can also refer to a protein or a nucleic acid that can be obtained using any means, for example, a genetic engineering technique, including recombinant DNA technology, or a chemical synthesis method, or the like, so long as the amino acid sequence of the protein or the nucleotide sequence of the nucleic acid thus obtained is identical to the amino acid sequence of the protein or the nucleotide sequence of the nucleic acid that exists naturally in, is expressed naturally in, and/or is produced naturally by the organism. The phrase “a protein” can include, but is not limited to, a peptide, oligopeptide, polypeptide, protein, enzyme, and so forth. The phrase “a nucleic acid” can include deoxyribonucleic acid (DNA) and ribonucleic acid (RNA), and can specifically include, but is not limited to, expression regulatory sequences, including promoters, attenuators, terminators, and the like, genes, intergenic sequences, nucleotide sequences encoding signal peptides, pro-moieties of proteins, artificial amino acid sequences, and so forth. For example, a gene can particularly be DNA.

The phrase “non-modified”, which can be used interchangeably or equivalently to the phrases “native”, “natural”, and “wild-type”, in reference to a gene (for example, “a non-modified gene”) and a protein (for example, “a non-modified protein”), can mean, respectively, a native gene and a native protein that exist naturally in, are expressed naturally in, and/or are produced naturally by an organism, specifically a non-modified strain of a bacterium. Examples of such an organism can include any organism having the corresponding gene or protein, and specific examples thereof can include, for example, a bacterium belonging to the family Enterobacteriaceae such as, for example, the E. coli MG1655 strain (ATCC 47076) and P. ananatis 13355 strain (FERM BP-6614). A non-modified gene can encode a non-modified protein.

The bacterium as described herein may further have, for example, one or more modifications exemplified below. Such modifications can be, for example, modifications for imparting or enhancing the ability to produce L-methionine. Modifications for constructing the bacterium as described herein can be performed in any order.

For example, the bacterium as described herein may be further modified to overexpress a rarD gene.

The rarD gene of P. ananatis encodes a chloramphenicol resistance permease RarD (BioCyc database, biocyc.org, accession ID: G1H69-3687; UniProtKB/Swiss-Prot database, accession No. A0A0H3L1X8; KEGG, Kyoto Encyclopedia of Genes and Genomes, entry No. PAJ_3332). The nucleotide sequence of the rarD gene native to P. ananatis is shown in SEQ ID NO: 44, and the amino acid sequence of the RarD protein encoded by this gene is shown in SEQ ID NO: 45.

That is, the rarD gene may be a gene, such as DNA, having the nucleotide sequence of SEQ ID NO: 44, and the RarD protein may be a protein having the amino acid sequence of SEQ ID NO: 45.

The rarD gene is not limited to the gene having the nucleotide sequence shown in SEQ ID NO: 44, but may include a gene, such as DNA, having the variant nucleotide sequence of SEQ ID NO: 44, provided that the gene encodes a RarD protein. Similarly, the RarD protein is not limited to the protein having the amino acid sequence shown in SEQ ID NO: 45, but may include a protein having the variant amino acid sequence of SEQ ID NO: 45, provided that the protein has a function of the RarD protein. The phrase “a protein has a function of a RarD protein” can mean that a protein has a function of the protein having the amino acid sequence shown in SEQ ID NO: 45 or 47. Examples of the function of a protein can include the activity of the protein. The phrase “a protein has a function of a RarD protein” can also mean that the protein functions as a chloramphenicol resistance permease.

Moreover, the rarD gene may encode the RarD protein, wherein the asparagine (Asn) residue at position 86 is replaced with an acidic amino acid residue, which may be the aspartic acid (Asp, D) residue (N86D mutation) or the glutamic acid (Glu, E) residue (N86E mutation), in the amino acid sequence of a wild-type RarD protein, so as to enhance further the production of L-methionine using the bacterium as described herein. The mutation at position 86 may particularly be the N86D mutation. The RarD protein having the mutation at position 86 is also referred to as “a mutant RarD protein”. The rarD gene encoding a mutant RarD protein is also referred to as “a mutant rarD gene”. The phrase “a wild-type RarD protein” can refer to a RarD protein not having the mutation at position 86. A rarD gene encoding a mutant RarD protein is also referred to as “a wild-type rarD gene”. Examples of the wild-type rarD gene can include the rarD gene native to P. ananatis and variants thereof provided that the variants do not have a mutation resulting in the mutation at position 86 of the encoded protein. Examples of the wild-type RarD protein can include the RarD protein native to P. ananatis and variants thereof provided that the variants do not have the mutation at position 86. In other words, the mutant rarD gene may be identical to any wild-type rarD gene, except that the mutant rarD gene has a mutation resulting in the mutation at position 86 of the encoded protein. Also, the mutant RarD protein may be identical to any wild-type RarD protein, except that the mutant RarD protein has the mutation at position 86. Specifically, the amino acid sequence of the mutant RarD protein can be as shown in SEQ ID NO: 47, that can be encoded by the mutant rarD gene having the nucleotide sequence shown in SEQ ID NO: 46. That is, the rarD gene, specifically the mutant rarD gene, may have the nucleotide sequence of SEQ ID NO: 46, and the RarD protein, specifically the mutant RarD protein, may have the amino acid sequence of SEQ ID NO: 47. The rarD gene, specifically the mutant rarD gene, may also have a variant nucleotide sequence of SEQ ID NO: 46, provided that the variant nucleotide sequence has a mutation resulting in the mutation at position 86 of the encoded protein. The RarD protein, specifically the mutant RarD protein, may also have a variant amino acid sequence of SEQ ID NO: 47, provided that the variant amino acid sequence has the mutation at position 86 of the encoded protein.

The phrase “the asparagine (Asn) residue at position 86 in the amino acid sequence of a wild-type RarD protein” in the amino acid sequence of any chosen wild-type RarD protein can refer to an amino acid residue corresponding to the Asn residue at position 86 in the amino acid sequence shown as SEQ ID NO: 45 in an alignment of the amino acid sequence of the chosen wild-type RarD protein and the amino acid sequence of SEQ ID NO: 45. That is, the phrase “position 86” does not necessarily indicate an absolute position in the amino acid sequence of a wild-type RarD protein, but indicates a relative position based on the amino acid sequence shown as SEQ ID NO: 45. For example, when one amino acid residue is deleted at a position on the N-terminus side of the Asn residue at position 86 in the amino acid sequence shown as SEQ ID NO: 45, the Asn residue originally at position 86 becomes the Asn residue at position 85 in the resulting amino acid sequence, but it is still regarded as “the Asn residue at position 86 in the amino acid sequence of a wild-type RarD protein”. Such alignment can be performed, for example, using known gene analysis software. Specific examples of such software include DNASIS produced by Hitachi Solutions, GENETYX produced by Genetyx, ClustalW opened to the public by DDBJ, and so forth (Elizabeth C. Tyler et al., Computers and Biomedical Research, 1991, 24(1):72-96; Barton G. J. et al., J. Mol. Biol., 1987, 198(2):327-337; Thompson J D et al., Nucleic Acid Res., 1994, 22(22):4673-4680).

The mutant rarD gene can be obtained by, for example, modifying the wild-type rarD gene so that the encoded protein has the mutation at position 86. The wild-type rarD gene to be modified can be obtained as described above, for example, by cloning from Pantoea bacteria having the wild-type rarD gene, or chemical synthesis. Modification of a gene can be performed by a known method. For example, by the site-specific mutagenesis method, an objective mutation can be introduced into a target site of DNA. Examples of the site-specific mutagenesis method include a method of using PCR (Higuchi, R., 61, in PCR Technology, Erlich, H. A. Eds., Stockton Press, 1989; Carter P., Methods Enzymol., 1987, 154, 382), and a method of using a phage (Kramer, W. and Frits, H. J., Methods Enzymol., 1987, 154, 350; Kunkel, T. A. et al., Methods Enzymol., 1987, 154, 367). Furthermore, the mutant rarD gene can also be obtained without using the wild-type rarD gene. For example, the mutant rarD gene may be directly obtained by chemical synthesis.

For example, the bacterium as described herein may be further modified to overexpress a cysteine synthase gene.

The phrase “a cysteine synthase gene” can refer to a gene encoding a cysteine synthase. The phrase “a cysteine synthase” can refer to a protein having cysteine synthase activity (EC 2.5.1.47). Examples of the cysteine synthase gene can include a cysMgene and a cysK gene. The cysMgene may encode a cysteine synthase B that can use thiosulfate as a substrate. The cysK gene may encode a cysteine synthase A that can use sulfide as a substrate. Specific examples of the cysteine synthase gene can include the cysMgene native to P. ananatis. The nucleotide sequence of the cysM gene native to P. ananatis is shown in SEQ ID NO: 5, and the amino acid sequence of the CysM protein encoded by this gene is shown in SEQ ID NO: 48.

That is, the cysteine synthase gene, such as the cysMgene, may be a gene, such as DNA, having the nucleotide sequence of SEQ ID NO: 5, and the cysteine synthase, such as the CysM protein, may be a protein having the amino acid sequence of SEQ ID NO: 48.

The cysteine synthase gene, such as the cysMgene, is not limited to the gene having the nucleotide sequence shown in SEQ ID NO: 5, but may include a gene, such as DNA, having the variant nucleotide sequence of SEQ ID NO: 5, provided that the gene encodes a cysteine synthase. Similarly, the cysteine synthase, such as the CysM protein, is not limited to the protein having the amino acid sequence shown in SEQ ID NO: 48, but may include a protein having the variant amino acid sequence of SEQ ID NO: 48, provided that the protein has cysteine synthase activity.

For example, the bacterium as described herein may be further modified to have a mutant metA gene.

The metA gene encodes a homoserine transsuccinylase (EC 2.3.1.46). The phrase “a mutant metA gene” can refer to a gene encoding a mutant MetA protein. The phrase “a mutant MetA protein” can refer to a MetA protein having the R34C mutation, which is a mutation wherein the arginine (Arg) residue at position 34 is replaced with cysteine (Cys) residue in the amino acid sequence of a wild-type MetA protein. The phrase “a wild-type metA gene” can refer to a gene encoding a wild-type MetA protein. The phrase “a wild-type MetA protein” can refer to a MetA protein not having the R34C mutation. Examples of the wild-type metA gene can include the metA gene native to P. ananatis and variants thereof provided that the variants do not have a mutation resulting in the R34C mutation of the encoded protein. Examples of the wild-type MetA protein can include the MetA protein native to P. ananatis and variants thereof provided that the variants do not have the R34C mutation. In other words, the mutant metA gene may be identical to any wild-type metA gene, except that the mutant metA gene has a mutation resulting in the R34C mutation of the encoded protein. Also, the mutant MetA protein may be identical to any wild-type MetA protein, except that the mutant MetA protein has the R34C mutation. The nucleotide sequence of the metA gene native to P. ananatis is shown in SEQ ID NO: 21, and the amino acid sequence of the MetA protein encoded by this gene is shown in SEQ ID NO: 27. Specifically, an example of the amino acid sequence of a mutant MetA protein can be as shown in SEQ ID NO: 29, which can be encoded by the mutant metA gene having the nucleotide sequence shown in SEQ ID NO: 28. That is, the mutant metA gene may be a gene, such as DNA, having the nucleotide sequence of SEQ ID NO: 28, and the mutant MetA protein may be a protein having the amino acid sequence of SEQ ID NO: 29. The mutant metA gene may also be a gene, such as DNA, having a variant nucleotide sequence of SEQ ID NO: 28, provided that the variant nucleotide sequence has a mutation resulting in the R34C mutation of the encoded protein. The mutant MetA protein may also be a protein having a variant amino acid sequence of SEQ ID NO: 29, provided that the variant amino acid sequence has the R34C mutation. The mutant MetA protein may be a homoserine transsuccinylase resistant to feedback inhibition by L-methionine. In other words, the mutant MetA protein may be a protein having homoserine transsuccinylase activity and resistant to feedback inhibition by L-methionine. The aforementioned descriptions concerning variants of the 1st gene and the 1st protein can be applied similarly to variants of the metA gene and the MetA protein. The aforementioned descriptions concerning the phrase “the asparagine (Asn) residue at position 86 in the amino acid sequence of a wild-type RarD protein” can be applied similarly to the phrase “the arginine (Arg) residue at position 34 in the amino acid sequence of a wild-type MetA protein”. The aforementioned descriptions concerning means for obtaining and introducing the mutant rarD gene can be applied similarly to means for obtaining and introducing the mutant metA gene.

For example, the bacterium as described herein may be further modified to attenuate expression of a metJ gene.

The metJ gene encodes a Met repressor, which may repress the expression of the methionine regulon and of enzymes involved in SAM synthesis. Examples of the metJ gene can include those native to the host bacterium, such as P. ananatis. The nucleotide sequence of the metJ gene native to P. ananatis is shown in SEQ ID NO: 16, and the amino acid sequence of the MetJ protein encoded by this gene is shown in SEQ ID NO: 49.

That is, the metJ gene may be a gene, such as DNA, having the nucleotide sequence of SEQ ID NO: 16, and the MetJ protein may be a protein having the amino acid sequence of SEQ ID NO: 49.

The metJ gene is not limited to the gene having the nucleotide sequence shown in SEQ ID NO: 16, but may include a gene, such as DNA, having the variant nucleotide sequence of SEQ ID NO: 16, provided that the gene encodes a Met repressor. Similarly, the MetJ protein is not limited to the protein having the amino acid sequence shown in SEQ ID NO: 49, but may include a protein having the variant amino acid sequence of SEQ ID NO: 49, provided that the protein functions as a Met repressor. The phrase “a protein functions as a Met repressor” can mean that a protein has a function of repressing the expression of the methionine regulon and of enzymes involved in SAM synthesis.

The phrase “a bacterium has been modified to attenuate expression of a metJ gene” can mean that the bacterium has been modified in such a way that in the modified bacterium, expression of a metJ gene is attenuated. The expression of a metJ gene can be attenuated due to, for example, inactivation of the gene.

The phrase “a metJ gene is inactivated” can mean that the modified gene encodes a completely inactive or non-functional protein as compared with the gene encoding a protein that has inorganic pyrophosphatase activity. It is also acceptable that the modified DNA region is unable to naturally express the gene due to deletion of a part of the gene or deletion of the entire gene, replacement of one base or more to cause an amino acid substitution in the protein encoded by the gene (missense mutation), introduction of a stop codon (nonsense mutation), deletion of one or two bases to cause a reading frame shift of the gene, insertion of a drug-resistance gene and/or transcription termination signal, or modification of an expression regulatory region such as promoters, enhancers, operators, attenuators and termination signals, anti-termination signals, ribosome-binding sites (RBS) and other expression control elements. Inactivation of the gene can also be performed, for example, by conventional methods such as a mutagenesis treatment using UV irradiation or nitrosoguanidine (N-methyl-N′-nitro-N-nitrosoguanidine), site-directed mutagenesis, gene disruption using homologous recombination, and/or insertion-deletion mutagenesis (Yu D. et al., Proc. Natl. Acad. Sci. USA, 2000, 97(11):5978-5983; Datsenko K. A. and Wanner B. L., Proc. Natl. Acad. Sci. USA, 2000, 97(12):6640-6645; Zhang Y. et al., Nature Genet., 1998, 20:123-128) based on “Red/ET-driven integration” or “Med/ET-mediated integration”.

The phrase “a bacterium has been modified to attenuate expression of a metJ gene” can also mean that the modified bacterium contains a region operably linked to the gene, including sequences controlling gene expression such as promoters, enhancers, attenuators and transcription termination signals, ribosome-binding sites (RBSs), and other expression control elements, which is modified so that the expression level of the metJ gene is decreased as compared with a non-modified strain; and other examples (see, for example, WO95/34672; Carrier T. A. and Keasling J. D., Biotechnol. Prog., 1999, 15:58-64). The phrase “operably linked” in reference to a gene can mean that the regulatory region(s) is/are linked to the nucleotide sequence of the gene in such a manner so that the expression of the gene can be attained (for example, enhanced, increased, constitutive, basal, antiterminated, attenuated, deregulated, decreased, or repressed expression), and/or mRNA of the gene and/or an amino acid sequence encoded by the gene (so-called expression product) can be produced as a result of expression of the gene.

The phrase “a bacterium has been modified to attenuate expression of a metJ gene” can also mean that the bacterium has been modified in such a way that in the modified bacterium, the expression level (that is, expression amount) of a metJ gene is attenuated as compared with a non-modified strain, for example, a wild-type or parental strain. A decrease in the expression level of a gene can be measured as, for example, a decrease in the expression level of the gene per cell, which may be an average expression level of the gene per cell. The phrase “the expression level of a gene” or “the expression amount of a gene” can mean, for example, the amount of an expression product of a gene, such as the amount of mRNA of the gene or the amount of the protein encoded by the gene. The bacterium may be modified so that the expression level of the metJ gene per cell is reduced to, for example, 50% or less, 20% or less, 10% or less, 5% or less, or 0% of that in a non-modified strain.

The phrase “a bacterium has been modified to attenuate expression of a mea gene” can also mean that the bacterium has been modified in such a way that in the modified bacterium, the total amount and/or the total activity of the corresponding gene product, that is, a Met repressor, is decreased as compared with a non-modified strain. A decrease in the total amount and/or the total activity of a protein can be measured as, for example, a decrease in the amount or activity of the protein per cell, which may be an average amount or activity of the protein per cell. The bacterium can be modified so that the amount or activity of a Met repressor per cell is decreased to, for example, 50% or less, 20% or less, 10% or less, 5% or less, or 0% of that in a non-modified strain.

Expression of a metJ gene can also be attenuated by, specifically, for example, replacing an expression control sequence of the gene, such as a promoter on the chromosomal DNA, with a weaker one. The strength of a promoter can be defined by the frequency of initiation acts of RNA synthesis. Examples of methods for evaluating the strength of promoters are described in Goldstein M. A. et al. (Goldstein M. A. and Doi R. H., Prokaryotic promoters in biotechnology, Biotechnol. Annu. Rev., 1995, 1:105-128), and so forth. Furthermore, it is also possible to introduce one or more nucleotide substitutions in a promoter region of the gene and thereby modify the promoter to be weakened as disclosed in WO0018935 A1. Furthermore, it is known that substitution of several nucleotides in the Shine-Dalgarno (SD) sequence, and/or in the spacer between the SD sequence and the start codon, and/or a sequence immediately upstream and/or downstream from the start codon in the ribosome-binding site greatly affects the translation efficiency of mRNA.

Expression of a metJ gene can also be attenuated by, specifically, for example, inserting a transposon or an insertion sequence (IS) into the coding region of the gene (U.S. Pat. No. 5,175,107) or in the region controlling gene expression, or by conventional methods such as mutagenesis with ultraviolet (UV) irradiation or nitrosoguanidine (N-methyl-N′-nitro-N-nitrosoguanidine, NTG). Furthermore, the incorporation of a site-specific mutation can be conducted by known chromosomal editing methods based, for example, on λRed/ET-mediated recombination (Datsenko K. A. and Wanner B. L., Proc. Natl. Acad. Sci. USA, 2000, 97(12):6640-6645).

The bacterium can have, in addition to the properties already mentioned, other specific properties such as various nutrient requirements, drug resistance, drug sensitivity, and drug dependence, without departing from the scope of the present invention.

2. Method

The method of producing L-methionine using a bacterium as described herein includes the steps of cultivating (also called culturing) the bacterium in a culture medium to allow L-methionine to be produced, excreted or secreted, and/or accumulated in the culture medium or in cells of the bacterium, or both, and collecting the L-methionine from the culture medium and/or the cells. The method may further include, optionally, the step of purifying L-methionine from the culture medium and/or the cells. L-methionine can be produced in such a form as described above. L-methionine can be produced particularly in a free form or as a salt thereof, or as a mixture of them. For example, sodium, potassium, ammonium, and the like salts or an inner salt such as zwitterion of L-methionine can be produced by the method. This is possible as amino acids can react under fermentation conditions with each other or a neutralizing agent such as an inorganic or organic acidic or alkaline substance in a typical acid-base neutralization reaction to form a salt that is the chemical feature of amino acids which is apparent to the person skilled in the art.

The cultivation of the bacterium, and collection, and, optionally, purification of L-methionine from the medium and the like may be performed in a manner similar to the conventional fermentation methods wherein an L-amino acid is produced using a microorganism. That is, the cultivation of the bacterium, and collection and purification of L-methionine from the medium and the like may be performed by applying conditions that are suitable for the cultivation of the bacterium, and appropriate for the collection and purification of an L-amino acid, which conditions are well-known to the persons of ordinary skill in the art.

The culture medium can be either a synthetic or natural medium such as a typical medium that contains a carbon source, a nitrogen source, a sulphur source, a phosphorus source, inorganic ions, and other organic and inorganic components as required. As the carbon source, saccharides such as glucose, sucrose, lactose, galactose, fructose, arabinose, maltose, xylose, trehalose, ribose, and hydrolysates of starches; alcohols such as ethanol, glycerol, mannitol, and sorbitol; organic acids such as gluconic acid, fumaric acid, citric acid, malic acid, and succinic acid; fatty acids, and the like can be used. As the nitrogen source, inorganic ammonium salts such as ammonium sulfate, ammonium chloride, and ammonium phosphate; organic nitrogen such as of soy bean hydrolysate; ammonia gas; aqueous ammonia; and the like can be used. Furthermore, peptone, yeast extract, meat extract, malt extract, corn steep liquor, and so forth can also be utilized. The medium may contain one or more types of these nitrogen sources. The sulphur source can include ammonium sulphate, magnesium sulphate, ferrous sulphate, manganese sulphate, thiosulfate, sulfide, and the like. The medium can contain a phosphorus source in addition to the carbon source, the nitrogen source and the sulphur source. As the phosphorus source, potassium dihydrogen phosphate, dipotassium hydrogen phosphate, phosphate polymers such as pyrophosphoric acid and so forth can be utilized. Vitamins such as vitamin B1, vitamin B2, vitamin B6, nicotinic acid, nicotinamide, vitamin B12, required substances, for example, organic nutrients such as nucleic acids such as adenine and RNA, amino acids, peptone, casamino acid, yeast extract, and the like may be present in appropriate, even if trace, amounts. Other than these, small amounts of calcium phosphate, iron ions, manganese ions, and so forth may be added, if necessary.

Cultivation can be performed under conditions suitable for cultivating a bacterium chosen for the use in the method for producing L-methionine or a salt thereof. For example, the cultivation can be performed under aerobic conditions for from 16 to 72 hours or for from 16 to 24 hours, the culture temperature during cultivation can be controlled within from 30 to 45° C. or within from 30 to 37° C., and the pH can be adjusted between 5 and 8 or between 6 and 7.5. The pH can be adjusted using an inorganic or organic acidic or alkaline substance such as urea, calcium carbonate, or ammonia gas.

After cultivation, L-methionine can be collected from the culture medium. Specifically, L-methionine present outside of cells can be collected from the culture medium. Also, after cultivation, L-methionine can be collected from cells of the bacterium. Specifically, the cells can be disrupted, a supernatant can be obtained by removing solids such as the cells and the cell-disrupted suspension (so-called cell debris), and then L-methionine can be collected from the supernatant. Disruption of the cells can be performed using, for example, methods that are well-known in the art, such as ultrasonic lysis using high frequency sound waves, or the like. Removal of solids can be performed by centrifugation or membrane filtration, for example. Collection of L-methionine from the culture medium or the supernatant etc. can be performed using conventional techniques such as concentration, crystallization, ion-exchange chromatography, medium or high pressure liquid chromatography, or a combination of these, for example.

### EXAMPLES

The present invention will be more precisely explained below with reference to the following non-limiting examples.

### Example 1. Construction of L-Methionine-Producing Strains

It is known that biosynthesis of L-methionine can be affected positively by deleting the metJ gene encoding a negative transcription regulator of methionine regulon, and desensitizing the homoserine O-succinyltransferase (MetA) to the feedback inhibition by methionine and SAM (S-adenosyl methionine) (Chattopadhyay M. K. et al., Control of methionine biosynthesis in Escherichia coli K12: a closer study with analogue-resistant mutants, J. Gen. Microbiol., 1991, 137(3):685-691; Usuda Y. and Kurahashi O., Effects of deregulation of methionine biosynthesis on methionine excretion in Escherichia coli, Appl. Environ. Microbiol., 2005, 71(6):3228-3234). Therefore, metJ gene was deleted and a mutant metA gene was obtained in a P. ananatis bacterium to construct a model L-methionine-producing strain C2691.

It is also known that biosynthesis of methionine from thiosulfate requires cysteine synthase B which is encoded by the cysM gene (Russian Patent No. 2458981 C2; Nakamura T. et al., Enzymatic proof for the identity of the S-sulfocysteine synthase and cysteine synthase B of Salmonella typhimurium, J. Bacteriol., 1984, 158(3):1122-1127). Therefore, the cysM gene was overexpressed in a P. ananatis bacterium to construct a model L-methionine-producing strain C2691 that is able to utilize thiosulfate as a sulphur source.

1.1. Construction of P. ananatis C2338 Strain (SC17(0)λattL-kan^{R}-λattR-Pnlp8sd22-cysM)

The P. ananatis SC17(0)λattL-kan^{R}-λattR-Pnlp8sd22-cysM strain (abbreviated as C2338) having a promoter region of cysM gene (SEQ ID NO: 5) replaced with cassette λattL-kan^{R}-λattR-Pnlp8sd22 was constructed using Red-dependent integration. For this purpose, P. ananatis SC17(0) strain (U.S. Pat. No. 8,383,372 B2, VKPM B-9246) was cultured in an LB liquid culture medium (Sambrook J. and Russell D. W., Molecular Cloning: A Laboratory Manual (3^{rd} ed.), Cold Spring Harbor Laboratory Press, 2001) overnight. Then, 1 mL of the cultured medium was inoculated to 100 mL of an LB liquid culture medium containing isopropyl β-D-1-thiogalactopyranoside (IPTG) at final concentration of 1 mM, and the cells were cultured at 32° C. for 3 hours with shaking (250 rpm). The microbial cells were collected and washed three times with 10% glycerol to obtain competent cells. An amplified λattL-kan^{R}-λattR-Pnlp8sd22 DNA fragment having a recombinant sequence of promoter region of cysM gene at both termini was obtained by PCR using the primers P1 (SEQ ID NO: 6) and P2 (SEQ ID NO: 7), and pMW118-attL-kan-attR-Pnlp8sd22 plasmid (SEQ ID NO: 8) as a template. The resulting DNA fragment was purified using Wizard PCR Prep DNA Purification System (Promega) and introduced into the competent cells using an electroporation method. The cells were cultured in the SOC culture medium (Sambrook J. and Russell D. W., Molecular Cloning: A Laboratory Manual (3^{rd} ed.), Cold Spring Harbor Laboratory Press, 2001) for 2 hours, then applied onto the LB plate containing 35 mg/L of kanamycin, and cultured at 34° C. for 16 hours. Emerging colonies were refined in the same culture medium. Then, a PCR analysis (TaKaRa Speed Star®; 40 cycles at 92° C. for 10 seconds, 56° C. for 10 seconds and 72° C. for 60 seconds) was performed using primers P3 (SEQ ID NO: 9) and P4 (SEQ ID NO: 10) to confirm that the promoter region of cysM gene on the chromosome was replaced with the λattL-kan^{R}-λattR-Pnlp8sd22 cassette. As a result, the P. ananatis SC17(0)λattL-kan^{R}-λattR-Pnlp8sd22-cysM strain (C2338) was obtained.

1.2. Construction of P. ananatis C2597 Strain (SC17(0)ΔmdeA::λattL-kan^{R}-λattR-Pnlp8sd22-cysM)

The P. ananatis SC17(0)ΔmdeA::λattL-kan^{R}-λattR-Pnlp8sd22-cysM strain (abbreviated as C2597) having replaced silent gene mdeA (SEQ ID NO: 11) with cassette λattL-kan^{R}-λattR-Pnlp8sd22-cysM was constructed using ?Red-dependent integration. For this purpose, P. ananatis SC17(0) strain was cultured in an LB liquid culture medium overnight. Then, 1 mL of the cultured medium was inoculated to 100 mL of an LB liquid culture medium containing IPTG at final concentration of 1 mM, and the cells were cultured at 32° C. for 3 hours with shaking (250 rpm). The microbial cells were collected and washed three times with 10% glycerol to obtain competent cells. An amplified λattL-kan^{R}-λattR-Pnlp8sd22-cysM DNA fragment having a recombinant sequence of mdeA gene at both termini was obtained by PCR using the primers P5 (SEQ ID NO: 12) and P6 (SEQ ID NO: 13), and chromosome isolated from the strain C2338 (Example 1.1) as a template. The resulting DNA fragment was purified using Wizard PCR Prep DNA Purification System (Promega) and introduced into the competent cells using an electroporation method. The cells were cultured in the SOC culture medium for 2 hours, then applied onto the LB plate containing 35 mg/L of kanamycin, and cultured at 34° C. for 16 hours. Emerging colonies were refined in the same culture medium. Then, a PCR analysis (TaKaRa Speed Star®; 40 cycles at 92° C. for 10 seconds, 56° C. for 10 seconds and 72° C. for 60 seconds) was performed using primers P7 (SEQ ID NO: 14) and P8 (SEQ ID NO: 15) to confirm that the mdeA gene on the chromosome was replaced with the λattL-kan^{R}-λattR-Pnlp8sd22-cysM cassette. As a result, the P. ananatis SC17(0)ΔmdeA::λattL-kan^{R}-λattR-Pnlp8sd22-cysM strain (C2597) was obtained.

1.3. Construction of P. ananatis C2603 strain (SC17ΔmdeA::λattL-kan^{R}-λattR-Pnlp8sd22-cysM)

Chromosome DNA was isolated from the strain C2597 (SC17(0) ΔmdeA::λattL-kan^{R}-λattR-Pnlp8sd22-cysM) using EdgeBio PurElute Bacterial Genomic kit according to manufacturer instructions. The resulting chromosome DNA was used for transformation of P. ananatis SC17 strain (FERM BP-11091). For this purpose, P. ananatis SC17 strain was cultured in an LB liquid culture medium overnight. Then, 1 mL of the cultured medium was inoculated to 100 mL of an LB liquid culture medium and the cells were cultured at 32° C. for 2 hours with shaking (250 rpm). The microbial cells were collected and washed three times with 10% glycerol to obtain competent cells. Chromosome DNA isolated from the strain C2597 (Example 1.2) was introduced into the competent cells using an electroporation method. The cells were cultured in the SOC culture medium for 2 hours, then applied onto the LB plate containing 35 mg/L of kanamycin, and cultured at 34° C. for 16 hours. Emerging colonies were refined in the same culture medium. Then, a PCR analysis was performed as described in Example 1.2 to confirm the replacement of mdeA gene. As a result, the P. ananatis SC17ΔmdeA::λattL-kan^{R}-λattR-Pnlp8sd22-cysM strain (abbreviated as C2603) was obtained.

1.4. Construction of P. ananatis C2614 Strain by Deletion of Kan Gene from C2603 Strain (SC17ΔmdeA::λattL-kan^{R}-λattR-Pnlp8sd22-cysM)

The kanamycin resistant gene (kan) was deleted from the C2603 strain using an RSF(TcR)-int-xis (US20100297716 A1) plasmid. RSF(TcR)-int-xis was introduced into C2603 strain by an electroporation method, and the cells were applied onto LB culture medium containing tetracycline (15 mg/L) and cultured at 30° C. to obtain C2603/RSF(TcR)-int-xis strain.

The resulting plasmid-harboring strain C2603/RSF(TcR)-int-xis was refined in the LB culture medium containing 15 mg/L of tetracycline and 1 mM IPTG to obtain single colonies. Then, a single colony was applied onto the culture medium containing 50 mg/L of kanamycin and cultured at 37° C. overnight with shaking (250 rpm). The kanamycin-sensitive strain was applied onto the LB culture medium containing 10% sucrose (by weight) and 1 mM IPTG and cultured at 37° C. overnight in order to delete the RSF(TcR)-int-xis plasmid from the strain. A colony that was sensitive to tetracycline was selected, and the corresponding strain was designated as C2614.

1.5. Construction of P. ananatis C2607 Strain (SC17(0)ΔmetJ::λattL-cat^{R}-λattR)

The P. ananatis SC17(0)ΔmetJ::λattL-cat^{R}-λattR strain (abbreviated as C2607) having deleted the mea gene (SEQ ID NO: 16) was constructed using),Red-dependent integration. For this purpose, P. ananatis SC17(0) strain was cultured in the LB liquid culture medium overnight. Then, 1 mL of the cultured medium was inoculated to 100 mL of the LB liquid culture medium containing IPTG at final concentration of 1 mM, and the cells were cultured at 32° C. for 3 hours with shaking (250 rpm). The microbial cells were collected and washed three times with 10% glycerol to obtain competent cells. An amplified λattL-cat^{R}-λattR DNA fragment having a recombinant sequence of metJ gene at both termini was obtained by PCR using the primers P9 (SEQ ID NO: 17) and P10 (SEQ ID NO: 18), and pMW118-attL-cat-attR plasmid (Minaeva N. I. et al., BMC Biotechnol., 2008, 8:63) as a template. The resulting DNA fragment was purified using Wizard PCR Prep DNA Purification System (Promega) and introduced into the competent cells using an electroporation method. The cells were cultured in the SOC culture medium for 2 hours, then applied onto the LB plate containing 35 mg/L of chloramphenicol, and cultured at 34° C. for 16 hours. Emerging colonies were refined in the same culture medium. Then, a PCR analysis (TaKaRa Speed Star®; 40 cycles at 92° C. for 10 seconds, 56° C. for 10 seconds and 72° C. for 60 seconds) was performed using primers P11 (SEQ ID NO: 19) and P12 (SEQ ID NO: 20) to confirm that the metJ gene on the chromosome was replaced with the λattL-cat^{R}-λattR cassette. As a result, the P. ananatis SC17(0)ΔmetJ::λattL-cat^{R}-λattR strain (C2607) was obtained.

1.6. Construction of P. ananatis C2634 Strain (C2614ΔmetJ:λattL-cat^{R}-λattR)

Chromosome DNA was isolated from the strain C2607 (SC17(0)λmetJ:λattL-cat^{R}-λattR) using EdgeBio PurElute Bacterial Genomic kit according to manufacturer instructions. The resulting chromosome DNA was used for transformation of C2614 strain (Example 1.4). For this purpose, P. ananatis C2614 strain was cultured in an LB liquid culture medium overnight. Then, 1 mL of the cultured medium was inoculated to 100 mL of an LB liquid culture medium and the cells were cultured at 32° C. for 2 hours with shaking (250 rpm). The microbial cells were collected and washed three times with 10% glycerol to obtain competent cells. Chromosome DNA isolated from the strain C2607 (Example 1.5) was introduced into the competent cells using an electroporation method. The cells were cultured in the SOC culture medium for 2 hours, then applied onto the LB plate containing 35 mg/L of chloramphenicol, and cultured at 34° C. for 16 hours. Emerging colonies were refined in the same culture medium. Then, a PCR analysis was performed as described in Example 1.5 to confirm the replacement of metJ gene. As a result, the P. ananatis C2614ΔmetJ:λattL-cat^{R}-λattR strain (C2634) was obtained.

1.7. Construction of P. ananatis C2605 Strain (SC17(0)attL-kan^{R}-attR-Ptac71φ10-metA)

The P. ananatis SC17(0)λattL-kan^{R}-λattR-Ptac71φ10-metA strain (abbreviated as C2605) having replaced promoter region of metA gene (SEQ ID NO: 21) with cassette λattL-kan^{R}-λattR-Ptac71φ10 was constructed using λRed-dependent integration. For this purpose, P. ananatis SC17(0) strain was cultured in an LB liquid culture medium overnight. Then, 1 mL of the cultured medium was inoculated to 100 mL of an LB liquid culture medium containing IPTG at final concentration of 1 mM, and the cells were cultured at 32° C. for 3 hours with shaking (250 rpm). The microbial cells were collected and washed three times with 10% glycerol to obtain competent cells. An amplified λattL-kan^{R}-λattR-Ptac71φ10 DNA fragment having a recombinant sequence of promoter region of metA gene at both termini was obtained by PCR using the primers P13 (SEQ ID NO: 22) and P14 (SEQ ID NO: 23), and pMW118-attL-kan-attR-Ptac71φ10 plasmid (SEQ ID NO: 24) as a template. The resulting DNA fragment was purified using Wizard PCR Prep DNA Purification System (Promega) and introduced into the competent cells using an electroporation method. The cells were cultured in the SOC culture medium for 2 hours, then applied onto the LB plate containing 35 mg/L of kanamycin, and cultured at 34° C. for 16 hours. Emerging colonies were refined in the same culture medium. Then, a PCR analysis (TaKaRa Speed Star®; 40 cycles at 92° C. for 10 seconds, 56° C. for 10 seconds and 72° C. for 60 seconds) was performed using primers P15 (SEQ ID NO: 25) and P16 (SEQ ID NO: 26) to confirm that the promoter region of metA gene on the chromosome of the strain SC17(0) was replaced with the λattL-kan^{R}-λattR-Ptac71φ10 cassette. As a result, the P. ananatis SC17(0)λattL-kan^{R}-λattR-Ptac71φ10-metA strain (C2605) was obtained.

1.8. Construction of P. ananatis C2611 Strain (SC17λattL-kan^{R}-λattR-Ptac71φ10-metA)

Chromosome DNA was isolated from the strain C2605 (SC17(0)λattL-kan^{R}-λattR-Ptac71φ10-metA) (Example 1.7) using EdgeBio PurElute Bacterial Genomic kit according to manufacturer instructions. The resulting chromosome DNA was used for transformation of SC17 strain. For this purpose, P. ananatis SC17 strain was cultured in an LB liquid culture medium overnight. Then, 1 mL of the cultured medium was inoculated to 100 mL of an LB liquid culture medium and the cells were cultured at 32° C. for 2 hours with shaking (250 rpm). The microbial cells were collected and washed three times with 10% glycerol to obtain competent cells. Chromosome DNA isolated from the strain C2605 introduced into the competent cells using an electroporation method. The cells were cultured in the SOC culture medium for 2 hours, then applied onto the LB plate containing 35 mg/L of kanamycin, and cultured at 34° C. for 16 hours. Emerging colonies were refined in the same culture medium. Then, a PCR analysis was performed as described in Example 1.7 to confirm the replacement of the promoter region of metA gene. As a result, the P. ananatis SC17λattL-kan^{R}-λattR-Ptac71φ10-metA strain (abbreviated as C2611) was obtained.

1.9. Construction of P. ananatis C2619 Strain (C2611ΔmetJ:λattL-cat^{R}-λattR)

Chromosome DNA was isolated from the strain C2607 (SC17(0)ΔmetJ:λattL-cat^{R}-λattR) (Example 1.5) using EdgeBio PurElute Bacterial Genomic kit according to manufacturer instructions. The resulting chromosome DNA was used for transformation of C2611 strain (Example 1.8). For this purpose, P. ananatis C2611 strain was cultured in an LB liquid culture medium overnight. Then, 1 mL of the cultured medium was inoculated to 100 mL of an LB liquid culture medium and the cells were cultured at 32° C. for 2 hours with shaking (250 rpm). The microbial cells were collected and washed three times with 10% glycerol to obtain competent cells. Chromosome DNA isolated from the strain C2607 introduced into the competent cells using an electroporation method. The cells were cultured in the SOC culture medium for 2 hours, then applied onto the LB plate containing 35 mg/L of chloramphenicol, and cultured at 34° C. for 16 hours. Emerging colonies were refined in the same culture medium. Then, a PCR analysis was performed as described in Example 1.5 to confirm that the replacement of metJ gene. As a result, the P. ananatis C2611ΔmetJ::λattL-cat^{R}-λattR strain (abbreviated as C2619) was obtained.

1.10. Selection of P. ananatis Strain Having a Mutant Allele of metA Gene Encoding Feedback Resistant MetA

The cells of the C2619 strain (SC17λattL-kan^{R}-λattR-Ptac71φ10-metA ΔmetJ:λattL-cat^{R}-λattR) were inoculated into 50 mL-flask containing an LB liquid culture medium up to OD₆₀₀ of 0.05 and cultured with aeration (250 rpm) at 34° C. for 2 hours. The exponentially growing cell culture of the strain at OD₆₀₀ of 0.25 was treated with N-methyl-N′-nitro-N-nitrosoguanidine (NTG) (final concentration 25 mg/L) for 20 minutes. The obtained culture was centrifuged, washed two times with fresh LB liquid culture medium and spread onto M9-agarized plate containing glucose (0.2%) and norleucine (600 g/L). Obtained mutant strains were tested for the ability to produce L-methionine. The strain having the highest ability to produce L-methionine was selected, and the nucleotide sequence of metA gene in that strain was determined. The sequence analysis revealed the mutation in the metA gene resulting in the replacement of the arginine (Arg) residue at position 34 with cysteine residue (R34C mutation) in the amino acid sequence of the wild-type MetA (SEQ ID NO: 27). The amino acid sequence of the mutant MetA protein having the R34C mutation is shown in SEQ ID NO: 29, and the nucleotide sequence of the mutant metA gene encoding the mutant MetA protein is shown in SEQ ID NO: 28. Thus, the P. ananatis SC17λattL-kan^{R}λattR-Ptac71φ10-metA(R34C)ΔmetJ:λattL-cat^{R}-λattR strain (abbreviated as C2664) was constructed.

1.11. Construction of P. ananatis C2669 Strain (C2634λattL-kan^{R}-λattR-Ptac71φ10-metA(R34C))

Chromosome DNA was isolated from the strain C2664 (SC17λattL-kan^{R}-λattR-Ptac71φ10-metA(R34C)ΔmetJ::λattL-cat^{R}-λattR) using EdgeBio PurElute Bacterial Genomic kit according to manufacturer instructions. The resulting chromosome DNA was used for transformation of C2634 strain (Example 1.6). For this purpose, P. ananatis C2634 strain was cultured in an LB liquid culture medium overnight. Then, 1 mL of the cultured medium was inoculated to 100 mL of an LB liquid culture medium and the cells were cultured at 32° C. for 2 hours with shaking (250 rpm). The microbial cells were collected and washed three times with 10% glycerol to obtain competent cells. Chromosome DNA isolated from the strain C2664 introduced into the competent cells using an electroporation method. The cells were cultured in the SOC culture medium for 2 hours, then applied onto the LB plate containing 35 mg/L of kanamycin, and cultured at 34° C. for 16 hours. Emerging colonies were refined in the same culture medium. Then, a PCR analysis was performed as described in Example 1.7 to confirm the replacement of the promoter region of metA gene. As a result, the P. ananatis C2634λattL-kan^{R}-λattR-Ptac71φ10-metA(R34C) strain (abbreviated as C2669) was obtained.

1.12. Construction of P. ananatis C2691 Strain by Deletion of Kan and Cat Genes from C2669 Strain (C2614ΔmetJ::λattL-cat^{R}-λattR λattL-kan^{R}-λattR-Ptac71φ10-metA(R34C))

The kanamycin and chloramphenicol resistant genes (kan and cat, correspondingly) were deleted from C2669 strain (Example 1.11) using an RSF(TcR)-int-xis plasmid. RSF(TcR)-int-xis was introduced into C2669 strain by an electroporation method, and the cells were applied onto LB culture medium containing tetracycline (15 mg/L) and cultured at 30° C. to obtain C2669/RSF(TcR)-int-xis strain.

The resulting plasmid-harboring strain C2669/RSF(TcR)-int-xis was refined in the LB culture medium containing 15 mg/L of tetracycline and 1 mM IPTG to obtain single colonies. Then, a single colony was applied onto the culture medium containing 50 mg/L of kanamycin and 35 mg/L chloramphenicol and cultured at 37° C. overnight with shaking (250 rpm). Strains sensitive to both kanamycin and chloramphenicol were applied onto the LB culture medium containing 10% sucrose (by weight) and 1 mM IPTG and cultured at 37° C. overnight in order to delete the RSF(TcR)-int-xis plasmid from the strain. A colony that was sensitive to tetracycline was selected, and the corresponding strain was designated as C2691. In summary, the C2691 strain is deficient in mdeA and metJ genes and has been introduced with cysM and metA(R34C) genes.

1.13. Construction of P. ananatis C3208 Strain (SC17(0)λattL-cat^{R}-λattR-Ptac71φ10-C)

The P. ananatis SC17(0)λattL-cat^{R}-λattR-Ptac71φ10-C strain (abbreviated as C3208) having a promoter region of PAJ_RS05335 gene (SEQ ID NO: 1; abbreviated as C gene) replaced with cassette λattL-cat^{R}-λattR-Ptac71φ10 was constructed using λRed-dependent integration. For this purpose, P. ananatis SC17(0) strain was cultured in an LB liquid culture medium overnight. Then, 1 mL of the cultured medium was inoculated to 100 mL of an LB liquid culture medium containing IPTG at final concentration of 1 mM, and the cells were cultured at 32° C. for 3 hours with shaking (250 rpm). The microbial cells were collected and washed three times with 10% glycerol to obtain competent cells. An amplified λattL-cat^{R}-λattR-Ptac71φ10 DNA fragment having a recombinant sequence of promoter region of C gene at both termini was obtained by PCR using the primers P17 (SEQ ID NO: 30) and P18 (SEQ ID NO: 31), and pMW118-attL-cat-attR-Ptac71φ10 (SEQ ID NO: 32) plasmid as a template. The resulting DNA fragment was purified using Wizard PCR Prep DNA Purification System (Promega) and introduced into the competent cells using an electroporation method. The cells were cultured in the SOC culture medium for 2 hours, then applied onto the LB plate containing 35 mg/L of chloramphenicol, and cultured at 34° C. for 16 hours. Emerging colonies were refined in the same culture medium. Then, a PCR analysis (TaKaRa Speed Star®; 40 cycles at 92° C. for 10 seconds, 56° C. for 10 seconds and 72° C. for 60 seconds) was performed using primers P19 (SEQ ID NO: 33) and P20 (SEQ ID NO: 34) to confirm that the promoter region of C gene on the chromosome of the strain SC17(0) was replaced with the λattL-cat^{R}-λattR-Ptac71φ1.0 cassette. As a result, the P. ananatis SC17(0)λattL-cat^{R}-λattR-Ptac71φ10-C strain (C3208) was obtained. Incidentally, the strain C3208 has PAJ_RS05340 gene (SEQ ID NO: 3; abbreviated as E2 gene) downstream of the C gene, so that these genes can be co-expressed.

1.14. Construction of C3293 Strain (SC17(0)ΔybhK::λattL-cat^{R}-λattR-Ptac71φ10-CE2)

The P. ananatis ΔybhK::λattL-cat^{R}-λattR-Ptac71v10-CE2 strain (abbreviated as C3293) having a silent gene ybhK (SEQ ID NO: 35) replaced with cassette λattL-cat^{R}-λattR-Ptac71φ10-CE2 which contains the genes PAJ_RS05335 (SEQ ID NO: 1; abbreviated as C gene) and PALRS05340 (SEQ ID NO: 3; abbreviated as E2 gene) under the control of a Ptac71φ10 promoter was constructed using λRed-dependent integration. For this purpose, P. ananatis SC17(0) strain was cultured in an LB liquid culture medium overnight. Then, 1 mL of the cultured medium was inoculated to 100 mL of an LB liquid culture medium containing IPTG at final concentration of 1 mM, and the cells were cultured at 32° C. for 3 hours with shaking (250 rpm). The microbial cells were collected and washed three times with 10% glycerol to obtain competent cells. An amplified λattL-cat^{R}-λattR-Ptac71φ10-CE2 DNA fragment having a recombinant sequence of ybhK gene at both termini was obtained by PCR using the primers P21 (SEQ ID NO: 36) and P22 (SEQ ID NO: 37), and chromosome isolated from the strain C3208 (Example 1.13) as a template. The resulting DNA fragment was purified using Wizard PCR Prep DNA Purification System (Promega) and introduced into the competent cells using an electroporation method. The cells were cultured in the SOC culture medium for 2 hours, then applied onto the LB plate containing 35 mg/L of kanamycin, and cultured at 34° C. for 16 hours. Emerging colonies were refined in the same culture medium. Then, a PCR analysis (TaKaRa Speed Star®; 40 cycles at 92° C. for 10 seconds, 56° C. for 10 seconds and 72° C. for 60 seconds) was performed using primers P23 (SEQ ID NO: 38) and P24 (SEQ ID NO: 39) to confirm that the ybhK gene on the chromosome was replaced with the λattL-cat^{R}-λattR-Ptac71φ10-CE2 cassette. As a result, the P. ananatis SC17(0)ΔybhK::λattL-cat^{R}-λattR-Ptac71φ10-CE2 strain (C3293) was obtained.

1.15. Construction of C3568 Strain (C2691ΔybhK::λattL-cat^{R}-λattR-Ptac71φ10-CE2)

Chromosome DNA was isolated from the strain C3293 (SC17(0)ΔybhK::λattL-cat^{R}-λattR-Ptac71φ10-CE2) using EdgeBio PurElute Bacterial Genomic kit according to manufacturer instructions. The resulting chromosome DNA was used for transformation of C2691 strain. For this purpose, P. ananatis C2691 strain (Example 1.12) was cultured in an LB liquid culture medium overnight. Then, 1 mL of the cultured medium was inoculated to 100 mL of an LB liquid culture medium and the cells were cultured at 32° C. for 2 hours with shaking (250 rpm). The microbial cells were collected and washed three times with 10% glycerol to obtain competent cells. Chromosome DNA isolated from the strain C3293 (Example 1.14) introduced into the competent cells using an electroporation method. The cells were cultured in the SOC culture medium for 2 hours, then applied onto the LB plate containing 35 mg/L of kanamycin, and cultured at 34° C. for 16 hours. Emerging colonies were refined in the same culture medium. Then, a PCR analysis was performed as described in Example 1.14 to confirm the replacement of ybhK gene. As a result, the P. ananatis C2691ΔybhK::λattL-cat^{R}-ΔattR-Ptac71φ10-CE2 strain (abbreviated as C3568) was obtained.

1.16. Overexpression of Mutant rarD Gene in P. ananatis Strains

First, pMIV-Pnlp, an expression vector containing a constitutive promoter of the nlpD gene native to E. coli, was constructed. A DNA fragment was obtained by PCR using the primers P25 (SEQ ID NO: 40) and P26 (SEQ ID NO: 41), and chromosomal DNA of E. coli strain MG1655 (ATCC No. 47076) as a template. The resulting DNA fragment was purified using an agarose gel electrophoresis and then isolated (Qiaquick Gel Extraction Kit, Qiagen). The DNA fragment was treated with the restriction enzymes PaeI and SalI (Fermentas) and cloned into the vector pMIV-5JS (RU2458981 C2) cleaved with PaeI/SalI. Thus, the pMIV-Pnlp vector was constructed.

Then, a wild-type rarD gene was obtained by PCR using the primers P27 (SEQ ID NO: 42) and P28 (SEQ ID NO: 43), and chromosomal DNA of the P. ananatis SC17(0) strain (FERM BP-11091) as a template. The resulting DNA fragment was purified and isolated as described above, treated with the restriction enzymes SalI and XbaI (Fermentas) and cloned into the vector pMIV-Pnlp cleaved with SalI/XbaI. Thus, the pMIV-Pnlp-rarD plasmid harboring the wild-type rarD gene was obtained.

P. ananatis SC17 strain was transformed with the pMIV-Pnlp-rarD plasmid, and the strain resistant to 1 g/L of α-methyl-DL-methionine was selected. Analysis of the nucleotide sequence of the rarD gene on the plasmid harbored by this resistant strain revealed the substitution of the “aac” codon encoding the asparagine (Asn, N) residue to the “gac” codon encoding the aspartic acid (Asp, D) residue at the positions from 256 to 258 in the nucleotide sequence of the wild-type rarD gene (SEQ ID NO: 44). This substitution in the nucleotide sequence of the wild-type rarD gene resulted in the replacement of the asparagine residue at position 86 with aspartic acid residue (N86D mutation) in the amino acid sequence of the wild-type RarD protein (SEQ ID NO: 45). The pMIV-Pnlp-rarD(N86D) plasmid harboring the mutant rarD gene (SEQ ID NO: 46) that encodes the mutant RarD protein (SEQ ID NO: 47) was isolated from the strain.

The pMIV-Pnlp-rarD(N86D) plasmid was introduced into P. ananatis C2691 and C3568 strains (Examples 1.12 and 1.15, accordingly) using a routine electroporation procedure. Thus, the P. ananatis C2691/pMIV-Pnlp-rarD(N86D) and C3568/pMIV-Pnlp-rarD(N86D) strains were constructed.

### Example 2. Production of L-Methionine

The P. ananatis C2691/pMIV-Pnlp-rarD(N86D) and C3568/pMIV-Pnlp-rarD(N86D) strains were each cultivated at 32° C. for 18 hours in LB liquid culture medium. Then, 0.2 mL of the obtained cultures were inoculated into 2 mL of a fermentation medium in 20×200-mm test tubes and cultivated at 32° C. for 48 hours on a rotary shaker at 250 rpm until glucose was consumed. The composition of the fermentation medium is shown in Table 1.

TABLE 1
Composition of the fermentation medium

| | Final |
|---|---|
| | concentra- |
| Component | tion (g/L) |
| Glucose | 40.0 |
| (NH₄)₂SO₄ | 15.0 |
| KH₂PO₄ | 1.5 |
| MgSO₄ × 7H₂O | 1.0 |
| Thiamine-HCl | 0.1 |
| CaCO₃ | 25.0 |
| LB medium | 4% (v/v) |

The fermentation medium was sterilized at 116° C. for 30 min, except that glucose and CaCO₃ were sterilized separately as follows: glucose at 110° C. for 30 min and CaCO₃ at 116° C. for 30 min. The pH was adjusted to 7.0 by KOH solution.

After cultivation, the amount of accumulated L-methionine was determined using Agilent 1260 amino-acid analyzer. The results of four independent test tube fermentations (as average values±standard deviations) are shown in Table 2. As one can see from the Table 2, the modified P. ananatis C3568/pMIV-Pnlp-rarD(N86D) strain was able to accumulate a higher amount (g/L) of L-methionine (Met) as compared with the parental P. ananatis C2691/pMIV-Pnlp-rarD(N86D) strain.

TABLE 2

| P. ananatis strain | Met, g/L |
|---|---|
| C2691/pMIV-Pnlp-rarD(N86D) (control strain) | 0.92 ± 0.03 |
| C3568 (C2691ΔybhK::λattL-cat^{R}-λattR-Ptac71φ10-CE2)/ | 1.22 ± 0.01 |
| pMIV-Pnlp-rarD(N86D) | |

In addition, it was separately confirmed that introduction of a plasmid carrying the CE2 genes into a P. ananatis L-methionine-producing strain without deletion of ybhK gene also results in an enhanced production of L-methionine (data not shown).

While the invention has been described in detail with reference to preferred embodiments thereof, it will be apparent to the one of ordinary skill in the art that various changes can be made, and equivalents employed, without departing from the scope of the invention.

### INDUSTRIAL APPLICABILITY

The method of the present invention is useful for the production of L-methionine by fermentation of a bacterium.

1. A method for producing L-methionine comprising:
(i) cultivating in a culture medium a bacterium which has an ability to produce L-methionine to produce and accumulate the L-methionine in the culture medium or cells of the bacterium, or both, and
(ii) collecting the L-methionine from the culture medium or the cells, or both,
wherein said bacterium has been modified to overexpress a 1st gene and a 2nd gene,
wherein said 1st gene is selected from the group consisting of:
(1A) a DNA comprising the nucleotide sequence shown in SEQ ID NO: 1,
(1B) a DNA encoding a protein comprising the amino acid sequence shown in SEQ ID NO: 2,
(1C) a DNA encoding a protein comprising the amino acid sequence shown in SEQ ID NO: 2, but wherein said amino acid sequence includes substitution, deletion, insertion, and/or addition of 1 to 30 amino acid residues, and wherein said DNA comprises a property that if the DNA is overexpressed in the bacterium, the amount of L-methionine produced by the bacterium is increased as compared with that observed for a non-modified strain,
(1D) a DNA encoding a protein comprising an amino acid sequence having an identity of not less than 85% with respect to the entire amino acid sequence shown in SEQ ID NO: 2, and wherein said DNA comprises a property that if the DNA is overexpressed in the bacterium, the amount of L-methionine produced by the bacterium is increased as compared with that observed for a non-modified strain,
(1E) a DNA comprising a nucleotide sequence that is able to hybridize under stringent conditions with a nucleotide sequence complementary to the sequence shown in SEQ ID NO: 1, and wherein said DNA comprises a property that if the DNA is overexpressed in the bacterium, the amount of L-methionine produced by the bacterium is increased as compared with that observed for a non-modified strain,
(1F) a DNA comprising a nucleotide sequence having an identity of not less than 85% with respect to the entire nucleotide sequence shown in SEQ ID NO: 1, and wherein said DNA comprises a property that if the DNA is overexpressed in the bacterium, the amount of L-methionine produced by the bacterium is increased as compared with that observed for a non-modified strain, and
(1G) a DNA comprising a variant nucleotide sequence of SEQ ID NO: 1 due to the degeneracy of the genetic code, and
wherein said 2nd gene is selected from the group consisting of:
(2A) a DNA comprising the nucleotide sequence shown in SEQ ID NO: 3,
(2B) a DNA encoding a protein comprising the amino acid sequence shown in SEQ ID NO: 4,
(2C) a DNA encoding a protein comprising the amino acid sequence shown in SEQ ID NO: 4, but wherein said amino acid sequence includes substitution, deletion, insertion, and/or addition of 1 to 30 amino acid residues, and wherein said DNA comprises a property that if the DNA is overexpressed in the bacterium, the amount of L-methionine produced by the bacterium is increased as compared with that observed for a non-modified strain,
(2D) a DNA encoding a protein comprising an amino acid sequence having an identity of not less than 85% with respect to the entire amino acid sequence shown in SEQ ID NO: 4, and wherein said DNA comprises a property that if the DNA is overexpressed in the bacterium, the amount of L-methionine produced by the bacterium is increased as compared with that observed for a non-modified strain,
(2E) a DNA comprising a nucleotide sequence that is able to hybridize under stringent conditions with a nucleotide sequence complementary to the sequence shown in SEQ ID NO: 3, and wherein said DNA comprises a property that if the DNA is overexpressed in the bacterium, the amount of L-methionine produced by the bacterium is increased as compared with that observed for a non-modified strain,
(2F) a DNA comprising a nucleotide sequence having an identity of not less than 85% with respect to the entire nucleotide sequence shown in SEQ ID NO: 3, and wherein said DNA comprises a property that if the DNA is overexpressed in the bacterium, the amount of L-methionine produced by the bacterium is increased as compared with that observed for a non-modified strain, and
(2G) a DNA comprising a variant nucleotide sequence of SEQ ID NO: 3 due to the degeneracy of the genetic code.

2. The method according to claim 1, wherein each of said 1st and 2nd genes is overexpressed by increasing the copy number of the gene, by modifying an expression regulatory region of the gene, or a combination thereof, so that the expression of said 1st and 2nd genes is enhanced as compared with a non-modified bacterium.

3. The method according to claim 1, wherein said bacterium is a bacterium belonging to the family Enterobacteriaceae.

4. The method according to claim 1, wherein said bacterium is a bacterium belonging to the genus Escherichia or Pantoea.

5. The method according to claim 1, wherein said bacterium is Escherichia coli or Pantoea ananatis.

6. The method according to claim 1, wherein said bacterium has been further modified to overexpress a rarD gene.

7. The method according to claim 6, wherein said rarD gene is overexpressed by increasing the copy number of the gene, by modifying an expression regulatory region of the gene, or a combination thereof, so that the expression of said rarD gene is enhanced as compared with a non-modified bacterium.

8. The method according to claim 1, wherein said bacterium has been further modified to overexpress a gene encoding cysteine synthase.

9. The method according to claim 8, wherein said gene encoding cysteine synthase is overexpressed by increasing the copy number of the gene, by modifying an expression regulatory region of the gene, or a combination thereof, so that the expression of said gene encoding cysteine synthase is enhanced as compared with a non-modified bacterium.

10. The method according to claim 8, wherein said gene encoding cysteine synthase is a cysM gene.

11. The method according to claim 1, wherein said bacterium has been modified further to comprise a metA gene encoding a MetA protein, wherein the amino acid sequence of the MetA protein has the amino acid substitution R34C.

12. The method according to claim 1, wherein said bacterium has been further modified to attenuate expression of a metJ gene.

13. The method according to claim 12, wherein said metJ gene is deleted.
